(19) United States
(12) Patent Application Publication
(10) Pub. No.: US 2026/0141994 A1
(43) Pub. Date: May 21, 2026

(54) SYSTEM AND METHOD FOR CLINICAL TRIAL SCENARIO PLANNING

(71) Applicant: Pharmaco Investments, Inc., Wilmington, CA (US)

(72) Inventors: Paulina Zelenay McAtee, Los Alamos, NM (US); Jeffrey A. Zimmerman, Marietta, GA (US); Dave Hiltbrand, Wilmington, NC (US); Caelin M. Quigley, New Braunfels, TX (US); David Berry, San Juan Capistrano, CA (US)

(21) Appl. No.: 19/393,421

(22) Filed: Nov. 18, 2025

Related U.S. Application Data
(60) Provisional application No. 63/721,829, filed on Nov. 18, 2024.

(51) Int. Cl. G16H 10/20

(52) U.S. Cl. CPC G16H 10/20

(57) ABSTRACT

The trial planning system of various embodiments herein may include a data warehouse configured to receive data from one or more data sources and interconnected with a modelling system and a forecasting system. Such a modelling system may include a pre-award module and a post-award module, each of which may comprise at least one model configured to generate a predictive trial output pertaining to the bid and planning phase and post-award phase of a clinical trial, respectively, including, without limitation, various metrics related to trial start-up, site activation, and patient recruitment. Such a forecasting system may include a variety of modules configured to enable a user to input various parameters relating to a trial, view and select sites for trial assessment, display various graphics details generated forecasts, and override the predictive trial outputs generated by the modelling system to develop more accurate, detailed, and granular forecasts for a given trial.

### CROSS-REFERENCE TO RELATED APPLICATIONS

This application claims the benefit of U.S. Provisional Application No. 63/721,829 filed Nov. 18, 2024, the entire disclosure of which is incorporated by reference.

### TECHNICAL FIELD

The present disclosure relates to interprogram and interprocess communications for computing systems and, more particularly, to data-processing systems for executing machine-learning pipelines, structural modeling operations, and predictive computation using distributed database components and multicomputer data-transfer mechanisms.

### BACKGROUND

Clinical trials are a lengthy and expensive process typically requiring over ten years and one billion dollars of investment to bring a single drug product to market. One factor driving the length and costs associated with clinical trials is the initial startup phase of a trial, including both site selection and patient recruitment, each of which play a vital role as to whether a given trial is performed timely, efficiently, and effectively. Indeed, poor site selection and/or patient recruitment may lead to start-up delays and poor-quality data, each of which can greatly delay trials and increase costs.

One factor driving the issues of site selection and patient recruitment stems from the uniqueness of clinical trials. Whether due to different drug products, different patient groups, different trials locales, or otherwise, each trial is different from another. As a result, there is no single, all-encompassing strategy for site selection and patient recruitment; rather, each trial demands a different approach. Naturally, reinventing the wheel of clinical trial startup can be a laborious manual process, and the lack of one-to-one reference points raises the potential for mistakes by even the most prepared and diligent clinical trial providers.

The foregoing issues are only exacerbated by the current inability to readily provide real-time assessments of the progress of clinical trial start-up processes. The inability to readily assess and remediate a trial startup plan renders clinical trial providers slow to react to unexpected issues and/or demands. And often, the decisions made by the providers can be based on inaccurate information, potentially leading to further issues down the line. Hence, mistakes often compound during the trial startup and recruitment phase, causing even further delays and costs.

Continued increases in trial complexity further complicate matters. Trials addressing unresolved medical needs often require more expansive and rigorous trial design, while greater and/or more stringent regulatory requirements further complicate matters. For instance, the need for greater patient diversity in clinical trials can impose complexity during the start-up and recruitment processes of a trial, such as by mandating a larger global scale or by intensifying the need for recruitment in highly specific subpopulations and/or regions. Similarly, more complex trials often necessitate greater involvement on the part of a patient and often leads to a decrease in patient retention. Such objectives, design methodologies, statistical considerations, regulatory requirements, and other aspects relating to the design, organization, and performance of a clinical trial are often outlined in a document referred to as a protocol.

Hence, there exists a need in the art for a solution directed to the foregoing issues. Such as solution should enable accurate forecasting of site selection, patient recruitment, and other procedures relevant to the start-up of clinical trials. Such a solution should further be configured for real-time data updates, thereby enabling greater access to data for clinical trial providers, as well as reforecasting solutions in view of already-planned start-up scenarios. Furthermore, such a solution should further enable clinical trial providers to manually configure the parameters for the aforementioned forecasting solution, thus enabling such providers greater flexibility in finding an appropriate solution for a given trial. And any such solution should be designed to enable clinical trial providers to have transparency to their forecasts, enabling easier visualization and selection into trial design planning.

### BRIEF SUMMARY

The trial planning system of various embodiments of the present invention may utilize a modelling system to generate one or more forecasts relevant to the site selection and patient recruitment procedures of clinical trial start-up. Such a trial planning system may generally comprise one or more data source(s) communicatively configured in connection with a data warehouse. Such a data warehouse may be configured to ingest raw datasets, such as historical clinical trial data, from such data source(s) and generate appropriate datasets for a modelling system interconnected therewith. Such a modelling system may be configured to generate one or more forecast outputs related to, for instance, site selection and/or patient recruitment. Such a trial planning system may further comprise a forecasting system configured in input/output communication with one or more user devices for interaction with the predictive trial output(s) and/or other relevant metrics generated by the modelling system.

The data source(s) of various embodiments of the present disclosure may comprise a variety of different sources having a variety of different data applicable to clinical trials. For instance, one such data source may include a trial management system, which may contain a variety of pieces of data related to previously conducted trials. Internal data sources and external data sources may similarly provide relevant data, whether associated with previously conducted clinical trials or otherwise.

As noted above, a data warehouse may be communicatively interconnected with such data sources for the receipt of data therefrom, such as through a data ingestion module. Such a data ingestion module may generally be configured to ingest and standardize data from such disparate sources, for the creation of a raw dataset therefrom. Such a raw dataset may include a variety of different information related to clinical trial start-up, site selection, and patient recruitment including, without limitation: (a) account and sponsor information, such as the name and information about a trial sponsor—i.e., the party initiating and financing a trial; (b) dates and timelines, such as the activation and recruitment dates for a trial, contract dates, protocol dates—i.e., when a protocol (the document defining how a trial will be performed) was finalized and contracted for—, enrollment and screening dates, and site qualification and close-out dates; (c) identifiers and codes, such as those relating to and identifying sites, institutions, and investigators—i.e., the medical professional performing the clinical studies; (d) status and flags, such as those identifying the status and conditions of a trial; (e) performance metrics, such as actual enrollment rates, actual screening rates, and deviations and/or disqualifications relating thereto; (f) contractual and regulatory information, such as details relating to contractual terms and regulatory compliance; (g) budget and financials, such as metrics identifying proposed budgets for a trial; (h) protocol and study design, such as information relating to the operation of the trial itself, including study conversion and target populations; (i) therapeutic areas, such as the indications and conditions related to a study; (j) visit and meeting information, such as the visits required to initiate a study for a patient; and (k) engineered features—i.e., various metrics derived from the foregoing—, such as number of patients screened per month and historical dropout rates. As may be understood, the foregoing enumerated features of the raw dataset are merely exemplary, and not comprehensive.

Once the raw dataset is ingested by the data warehouse, such a raw dataset may be transformed into a dataset applicable for use by the modelling system referenced heretofore and discussed in greater detail hereafter. Specifically, such a data warehouse may include a historical feature module which may transform the raw dataset into a historical feature dataset. Such a historical feature module may utilize a variety of processes to effectuate such a transformation. For instance, such a historical feature model may aggregate particular features of the raw dataset, such as by portions thereof grouped into a specific category, and utilize various statistical functions—e.g., mean, median, etc.—to develop the historical feature dataset. In at least some embodiments, a rolling join may be utilized by the historical feature model to aggregate and/or group features of the raw dataset, such as between specific start and end dates. Such a historical feature model may additionally include a just-in-time compiler, such as Numba, to accelerate application of the same on a large raw dataset.

In view thereof, the historical feature module of at least one embodiment of the present invention may transform the raw dataset into a historical feature dataset comprising a plurality of engineered features, at least some of which may be segmented according to other variables. For instance, and without limitation, such historical feature dataset may include: (a) enrollment rate metrics; (b) first site qualified metrics; (c) pre-study visit metrics; (d) study closed metrics; (e) days between site activation to first patient enrolled metrics; (f) days from ethics committee approval to eligible for activation metrics; (g) days from award to first site qualified metrics; (h) randomized contracted patients metrics; (i) days from eligible for activation to recruiting metrics; (j) days from eligible for participation to first site qualified metrics; (k) recruitment days metrics; (l) days from first site qualified to qualification metrics; (m) days from potential to eligible for participation metrics; (n) days from potential to eligible for participation metrics; (n) days from qualification to any recruiting metrics; (o) days from qualification to submission metrics; (p) days from request for proposal to first site qualified metrics; (q) days from request for proposal to potential metrics; (r) significant deviation metrics; (s) days from submission to approval metrics; (t) days from submission to approval metrics; (u) pre-selection visit status metrics; (v) screen-fail ratio metrics; (w) screening rate metrics; (x) dropout ratio metrics; (y) business code metrics; (z) non-enrollment metrics; and (aa) study country weekly ramp rate metrics. As previously noted, the foregoing metrics of the historical feature dataset may be segmented according to a variety of historical feature variables, such as, country, indication, therapeutic area, sponsor, phase, and institution, amongst others. As may be understood, the foregoing enumerated features and/or variables of the historical feature dataset are merely exemplary, and non-limiting.

Once the historical feature dataset is generated, a preprocessing module of the data warehouse may generate a training dataset and a testing dataset therefrom. In at least one embodiment, such a preprocessing module may utilize a procedure, such as a train-test-split procedure, to divide the historical feature dataset into a training dataset and a testing dataset. Such a preprocessing module may additionally generate an inference dataset from, for instance, data distribution metrics and/or performance metrics received by the monitoring module of the data warehouse from the modelling system. Such an inference dataset may be batched daily, and may include, for instance, pre-qualified metrics, pre-feasibility metrics, and post-qualified metrics. In so doing, the trial planning system of at least one embodiment of the present disclosure may be configured to compute every combination of investigator, institution, and therapeutic area in relation to the historical feature dataset, thereby covering every clinical trial site necessitating a predictive trial output which, as may be understood, may cover millions of records.

As previously noted, the data warehouse of various embodiments of the present disclosure may be communicatively configured in connection with a modelling system. Such a modelling system may comprise one or more machine learning models configured to, whether individually and/or collectively, generate a predictive trial output relating to, for instance, clinical trial site selection, patient recruitment, and other initiation and/or activation procedures related to clinical trial start-up.

In at least one embodiment, such a modelling system may comprise a pre-award module and a post-award module. Such a pre-award module may be configured to generate a predictive trial output related to the bid and planning phase of a clinical trial, whereas such a post-award module may be configured to generate a predictive trial output related to the phase of a clinical trial after a study is awarded. Thus, as may be understood, the predictive trial output of the post-award module may be based on, for instance, actual data related to the status of the startup procedures for a in-process clinical trial. For the sake of clarity, the outputs of the pre-award module and post-award module, whether individually or collectively, may be referred to as a predictive trial output herein; however, it may be understood a pre-award predictive trial output may refer solely to the output of the pre-award module, whereas a post-award predictive trial output may refer solely to the output of the post-award module.

The pre-award module of at least one embodiment of the present invention may include one or more machine learning models each directed to predict a different feature value. More specifically, the pre-award module of at least one embodiment may include six discrete models directed to different outputs, namely: (a) a qualification model generating a qualification metric; (b) an activation model generating an activation metric; (c) an activated quantity model generating an activated quantity metric; (d) a screening model generating a screening metric; (e) a screen-fail model generating a screen-fail metric; and (f) a dropout model generating a dropout metric. Each of these models will be discussed in greater detail hereafter.

The foregoing models of the pre-award module may, in at least one embodiment of the present invention, utilize a gradient boosted decision trees model (for example, a model built using gradient boosting where the base learners are classification and regression trees), such as XGBoost, although other similar methodologies and/or models are contemplated herein. More particularly, the activation model and the activated quantity model may each utilize XGBoost, the qualification model, screening model, and screen-fail model may each utilize XGBoost with a Yeo-Johnson transform to handle more flexible skewing and stabilize variance, and the droput model may utilize XGBoost with a log transformation, such as a log(1)p transformation, to positively skew the distribution—i.e., generate a distribution having a long tail on the right side thereof. The post-award module, meanwhile, may comprise one or more sequence-based deep learning models, such as a long short term memory network, a convolutional neural network, and/or transformers configured to predict, whether alone or in combination, a sequence of patient counts each month using the actual recruitment, screening, enrollment, and/or dropout metrics obtained by a given trial site and/or clinical trial after the award and activation thereof.

The modelling system of at least one embodiment of the present invention may further comprise a validation module configured to analyze the performance of the various models of the pre-award module and/or post-award module, as well as train and/or tune the same. Hence, it may be understood such a validation module may comprise an evaluation component, configured to evaluate the performance, and a training component, configured to tune and train the models. In at least one embodiment, the various models of the pre-award module may undergo a parallel training and evaluation procedure. Specifically, each of the models of the pre-award module—i.e., the qualification model, the activation model, the activated quantity model, the screening model, the screen-fail model, and the dropout model—may each be trained using the training dataset by the training component, evaluated using the testing dataset by the evaluation component, and the trained again using the training dataset by the training component. In so doing, the models of the pre-award module, for instance, may get an accurate assessment of the error generalization by the evaluation component, which may be resolved during the subsequent training by the training component.

The output of the modelling system—e.g., the predictive trial output—may pass through the data warehouse before transmission to the forecasting system communicatively interconnected therewith. Such a forecasting system may be configured for access through a web browser installed on a user device, and may be configured as a web application, a software-as-a-service, or some other similar such means as understood by those having skill in the art. Such a forecasting system may be configured in input-output communication with one or more user devices, thereby enabling users, such as those performing the forecasting operations of the trial planning system described herein, to input data relating to the clinical trial, generate one or more predictive trial outputs relating thereto, and generate visualization(s) and/or other deliverables relating to the forecasting of the clinical trial startup phase. Likewise, the forecasting system of at least one embodiment of the present invention may be configured to enable the user(s) to override one or more aspects of the predictive trial output, thereby enabling the user(s) to more particularly tailor the forecast(s) present therein to the specifics of the clinical trial at hand.

In view thereof, the forecasting system of various embodiments of the present invention may include a strategy forecasting module and a site selection module, each of which may enable a user to input and/or select data relevant to a clinical trial. Such a site selection module, for instance, may comprise a catalog of clinical trial sites, such as those known within the system and/or identified within the raw dataset. Accordingly, through such a site selection module, a user may be presented with a list of sites and other suitable data relating to those sites, thereby enabling an easier, more efficient site and country selection process.

Such a strategy forecasting module, meanwhile, may enable a user to input a variety of data relevant to the clinical trial at hand. For instance, such a strategy forecasting module may include a study target component, through which a user may input consideration parameters and/or milestone parameters relevant to a clinical trial. Such consideration parameters may include certain study-specific considerations including, without limitation, a target study start date, the number of patients to be involved in a trial, a preferred screening window length, an preferred enrollment period length, the duration for treatment and follow-up procedures, and potentially other seasonality factors relevant to a clinical trial—i.e., considerations which take into account the propensity for certain diseases, whether seasonal or otherwise, to present indications exhibiting seasonal variation. Such milestone parameters, meanwhile, may include start-up and/or enrollment milestones, including, without limitation, the target first site qualification, the target first site activation, the target last site activation, the target first subject screened, the target first subject enrolled, the target last subject screened, the target last subject enrolled, and the target last subject's last visit. Accordingly, it may be understood the strategy forecasting module may enable a user to define the parameters, considerations, and/or milestones applicable for a given trial, and makes changes thereto at some later time, thereby enabling greater configurability to the scenario planning of the trial planning system described herein.

The forecasting system of at least one embodiment of the present invention may additionally comprise a reforecasting module. Such a reforecasting module may be configured to enable save and recall previously saved forecasts, such as the predictive trial output(s), and edit those forecasts and/or compare the same with alternative forecasts. Thus, it may be understood such a reforecasting module may similarly utilize the study target component discussed heretofore to adjust the consideration parameters and/or milestone parameters used in a given forecast. Further, the reforecasting module of at least one embodiment of the present invention may additionally include a diversity component, through which a user may input applicable patient diversity parameters applicable for a given trial—e.g., sex diversity metrics, age diversity metrics, Hispanic diversity metrics, race allocation metrics, and gender allocation metrics. In so doing, study insight and depth of forecasting may be increased, thereby enabling more accurate results.

In at least one embodiment, such a forecasting system may additionally comprise a visualization module. Such a visualization module may receive the inputs of the strategy forecasting module and/or the reforecasting module and the apply the same in connection with the predictive trial output(s) received from the modelling system to generate one or more visualizations relating to the forecast at issue. For instance, such a visualization module may generate one or more plots, such as a study plot, a region plot, and a country plot, to depict a forecast at different levels of depth. And such a visualization module utilizes a site activation component to generate a site activation curve applicable and/or an enrollment component to generate a cumulative enrollment curve, each of which may be applicable to one or more of the foregoing plots. Meanwhile, an overlay component may be utilized to overlay one or more scenarios together, such as by utilizing one stored predictive trial output in connection with different consideration parameters, milestone parameters, and/or diversity parameters, thereby enabling a user to visualize how any one decision may impact a forecast.

In view thereof, it may be understood at least one embodiment of the present invention may comprise a system for clinical trial operational forecasting comprising: a data warehouse communicatively configured in connection with at least one data source, a modelling system, and a forecasting system; the data warehouse configured to receive a raw dataset from the at least one data source and generate at least one historical feature dataset from the raw dataset; the modelling system configured to generate at least one predictive trial output according to the at least one historical feature dataset; and the forecasting system configured to receive the at least one predictive trial output and generate at least one visualization therefrom, the at least one visualization depicting a site activation curve and an enrollment curve.

Likewise, an additional embodiment of the present invention may comprise a system for clinical trial operational forecasting comprising: a data warehouse communicatively configured in connection with at least one data source, a modelling system, and a forecasting system; the data warehouse configured to receive a raw dataset from the at least one data source; the data warehouse comprising a historical feature module configured to generate an historical feature dataset from the raw dataset; the modelling system comprising a pre-award module, the pre-award module comprising: a qualification model configured to predict a qualification metric for at least one site; an activation model configured to predict an activation metric for the at least one site; an activated quantity model configured to predict an activated quantity metric for the at least one site; a screening model configured to predict a screening metric for the at least one site; a screen-fail model configured to predict a screen-fail metric for the at least one site; a dropout model configured to predict a dropout metric for the at least one site; the modelling system configured to generate at least one predictive trial output from the pre-award module; the forecasting system configured to receive the least one predictive trial output, the forecasting system comprising: a strategy forecasting module configured receive at least one consideration parameter and at least one milestone parameter from at least one user device; a reforecasting module configured to receive at least one patient diversity parameter from the at least one user device; and a visualization module configured to generate at least one visualization according to the at least one consideration parameter, the at least one milestone parameter, the at least patient diversity parameter, and the at least one predictive trial output.

Further, yet an additional embodiment of the present invention may comprise a system for clinical trial operational forecasting comprising: a data warehouse communicatively configured in connection with at least one data source, a modelling system, and a forecasting system; the data warehouse configured to receive a raw dataset from the at least one data source; the data warehouse comprising a historical feature module configured to generate an historical feature dataset from the raw dataset; the modelling system comprising a pre-award module and a post-award module; the pre-award module comprising at least one pre-award model configured to generate at least one pre-award predictive trial output; the post-award module comprising at least post-award model configured to generate at least one post-award predictive trial output, the at least one post-award predictive trial output comprising a sequence of patient counts for each month according to real-time data collected from the at least one data source; the forecasting system configured to receive the at least one pre-award predictive trial output and the at least one post-award predictive trial output, the forecasting system comprising: a visualization module configured to generate at least one visualization, the at least one visualization configured to overlay the at least one pre-award predictive trial output and the at least one post-award predictive trial output.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A depicts a block diagram of a trial planning system, in accordance with at least one embodiment of the present invention.

FIG. 1B depicts a block diagram of a trial planning system, in accordance with at least one embodiment of the present invention.

FIG. 1C depicts a block diagram of a user device, in accordance with at least one embodiment of the present invention.

FIG. 2A depicts a block diagram of a data flow between a data warehouse and a modelling system, in accordance with at least one embodiment of the present invention.

FIG. 2B depicts a block diagram of a method of training a pre-award module, in accordance with at least one embodiment of the present invention.

FIG. 3A depicts a block diagram of a forecasting system, in accordance with at least one embodiment of the present invention.

FIG. 3B depicts a block diagram of a strategy forecasting module, a visualization module, and a reforecasting module of the forecasting system, in accordance with at least one embodiment of the present invention.

FIG. 3C depicts a visualization including a predictive trial output and generated by a visualization module, in accordance with at least one embodiment of the present invention.

FIG. 3D depicts a visualization including a study plot comprising at least one study activation curve and at least one enrollment curve, and generated by a visualization module, in accordance with at least one embodiment of the present invention.

FIG. 3E depicts a visualization including a region plot comprising at least one study activation curve and at least one enrollment curve, and generated by a visualization module, in accordance with at least one embodiment of the present invention.

FIG. 3F depicts a visualization including a country plot comprising at least one study activation curve and at least one enrollment curve, and generated by a visualization module, in accordance with at least one embodiment of the present invention.

FIGS. 4 and 5 illustrate a block diagram depicting data flow between components of the trial planning system of FIGS. 1A and 1B during execution of a process for generating, updating, and/or optimizing clinical-trial forecasts using historical data, predictive models, user inputs, and/or real-world operational data.

FIGS. 6, 7, 8, 9, 11, 12, 13, 14, 15, 16, and 17 are diagrams illustrating aspects of a graphical user interface generated by the a optimization module, according to some examples.

FIG. 10 illustrates a notification email generated by a site optimization module, according to some examples.

### DETAILED DESCRIPTION

Various embodiments of the present invention disclose a trial planning system 10 configured to generate a predictive trial output 228, wherein such predictive trial output 228 may be used in connection with user-input consideration parameters 328, milestone parameters 330, and/or patient diversity parameters 340 to generate one or more visualizations of one or more forecasts relating to the start-up procedures of a clinical trial, including both site selection and patient recruitment. In so doing, it may be understood the trial planning system disclosed herein may enable real-time scenario planning, improve site selection, and provide real-time, configurable forecasts of clinical trial start-up procedures, thereby improving clinical trial efficiency and reducing the costs associated therewith.

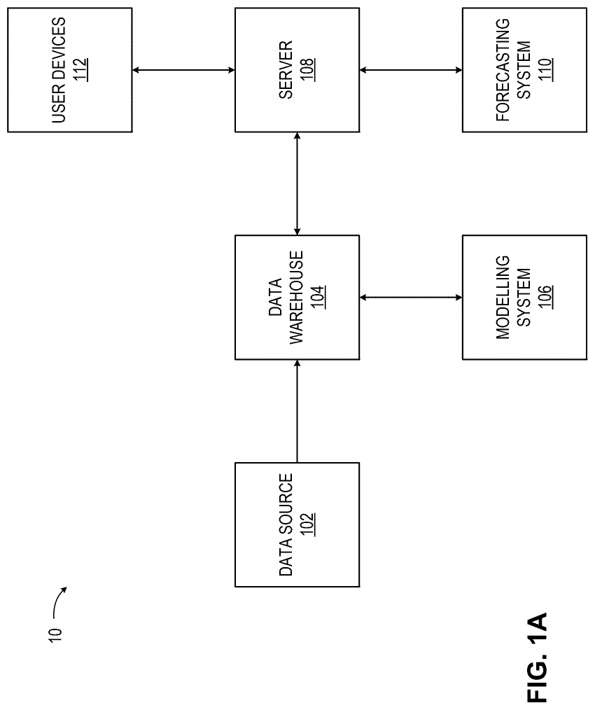

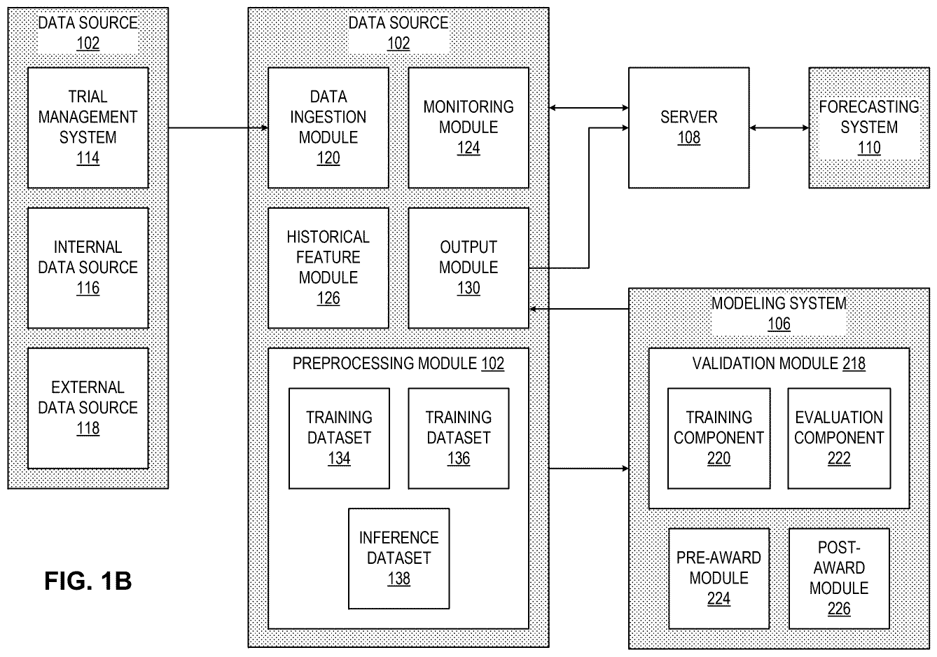

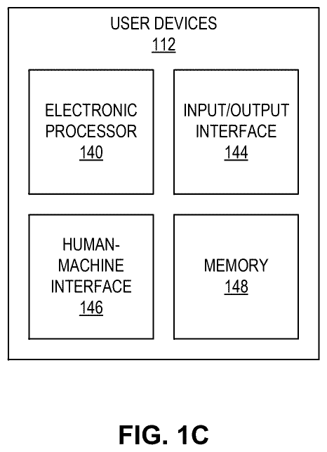

Depicted in FIG. 1A is a block diagram of at least one embodiment of the trial planning system 10 disclosed herein. As may be seen, such a trial planning system 10 may comprise a date data warehouse 104 communicatively placed in connection with a data source 102, a modelling system 106, and a server 108, through which a forecasting system 110 and one or more user devices 112 may be communicatively interconnected as well. Such communicative interconnection may occur via, for instance, the internet, some other large area network, peer-to-peer network, or some other similar means.

As noted above, the data warehouse 104 may be interconnected with one or more data sources 102. As shown in FIG. 1B, such data sources 102 may include, for instance, a trial management system 114, an internal data source 116, and an external data source 118. Such a trial management system 114 may comprise, without limitation, some clinical trial software configured to manage the various process, operations, and data involved in clinical studies. Hence, such a trial management system may store, and transmit to the data warehouse 104, data relating to trial sites, investigators, patients, therapeutic areas. Such an internal data source 116, meanwhile, may comprise some other data source owned, operated, and/or licensed by the provider of the trial planning system 10 described herein. For instance, such an internal data source 116 may comprise an internal system offered by another entity, such as Salesforce. Such an external data source 118, meanwhile, may comprise some other data source owned and/or operated by a third-party, such as a data repository licensed by the owner and/or operator of the trial planning system described herein.

With continued reference to FIGS. 1A and 1B, it may be seen the data sources 102 may transmit data to a data warehouse 104. Such a data warehouse 104 may comprise, for instance, a system configured to collect and aggregate data from a plurality of sources—i.e., the data sources 102 described herein—within a single, central location. Such a data warehouse 104 may comprise an internal data storage location or may instead be some system owned and operated by a third-party, such as a cloud-based data storage solution. For instance, in at least one embodiment of the present invention, such a data warehouse 104 may comprise the data cloud platform operated by Snowflake, Inc. ; however, it may be understood alternative such data storage solutions are contemplated herein.

Such collection and aggregation of data from the data sources 102 may occur through a data ingestion modules 120, which may standardize such data and create a raw dataset 122 therefrom. As previously discussed herein, such a raw dataset 122 may comprise a variety of different information generally related to clinical trial start-up, site selection, site activation, and patient recruitment procedures. For instance, and without limitation, such raw dataset 122 may include account and sponsor information, dates and timelines, identifiers and codes, status and flags, performance metrics, contractual and regulatory information, budget and financials, protocol and study design, therapeutic areas, visit and meeting information, and other engineered features. As may be understood, such a raw dataset may, in at least one embodiment, comprise a uniform format and/or structure, and may be commonly stored within the data warehouse 104.

Such a raw dataset 122 may subsequently be transformed into a historical feature dataset 128 via a historical feature module 126. In at least one embodiment, such a historical feature module 126 may be configured to aggregate one or more features of the raw dataset, group such features according to one or more categories, and apply one or more statistical functions to develop such historical feature dataset 128 from the raw dataset 122. As previously noted, such historical feature dataset 128 may comprise a variety of metrics including, without limitation: enrollment rate metrics, first site qualified metrics, pre-study visit metrics, study closed metrics, days between site activation to first patient enrolled metrics, days from ethics committee approval to eligible metrics, days from award to first site qualified metrics, randomized contracted patients metrics, days from eligible for activation to recruiting metrics, days from eligible for participation to first site qualified metrics; (k) recruitment days metrics, days from first site qualified to qualification metrics, days from potential to eligible for participation metrics, days from potential to eligible for participation metrics, days from qualification to any recruiting metrics, days from qualification to submission metrics, days from request for proposal to first site qualified metrics, days from request for proposal to potential metrics, significant deviation metrics, days from submission to approval metrics, days from submission to approval metrics, pre-selection visit status metrics, screen-fail ratio metrics, screening rate metrics, dropout ratio metrics, business code metrics, non-enrollment metrics, and study country weekly ramp rate metrics

As an example, it may be understood the historical feature dataset 128 may generate such screening rate metrics from one or more portions of the raw dataset. For instance, the dates and timelines category of the raw dataset may include dates identifying the enrollment, screening, and randomization of patients. Likewise, the category of the raw dataset 122 relating to performance metrics may include information relating to the enrollment and screening rates of patient enrollment and screening, such as the number of patients enrolled and/or screened, and the screen-fail rates for those patients. The engineered features of the raw dataset 122, meanwhile, may be configured to calculate other screening-related data, such as the number of patients screened per month at a given clinical trial site. In view thereof, it may be understood the historical feature dataset 128 may be configured to aggregate all the data relevant to the screening rate metrics thereof. Then, such historical feature dataset 128 may group all such data according to one or more feature categories. In the case of the screening rate metrics, such feature categories may include, without limitation, indication, country, institution ID, phase, therapeutic area, country and therapeutic area, country and indication, institution ID and indication, institution ID and phase, and institution ID and therapeutic area. As may be seen, such data may be grouped according to one or more categories. Then, such a historical feature dataset 128 may subsequently apply one or more statistical functions to those aggregated and grouped portions of the raw dataset 122. In the screening rate metrics example discussed above, the country category, for instance, may have one or more statistical functions applied thereto to identify the mean, median, standard deviation, and skewness of the data within that group. Meanwhile, the group for country and therapeutic area may instead be utilized to identify only the mean and median for such dataset. Thus, it may be understood the historical feature dataset 128 may be configured to identify different types of data for different types of historical features. And, as previously noted, such historical feature dataset may utilize a rolling join to further implement the above process, such as between specific start and end dates, thereby further engineering the historical feature dataset 128 to generate the most useful set of data.

With continued reference to FIGS. 1A and 1B, the data warehouse 104 of at least one embodiment of the present invention may utilize a preprocessing module 132 to generate a training dataset 134 and a testing dataset 136 from such historical feature dataset 128. For instance, such a preprocessing module may apply a train-test-split procedure to divide such historical feature dataset 128 into such training dataset 134 and testing dataset 136. The inference dataset 138 of the preprocessing module 132, in contrast, may instead be generate according to one or more metrics, such as data distribution metrics and/or performance metrics, received by the monitoring module 124 of the data warehouse 104, as will be discussed in greater detail hereafter.

As shown in FIG. 1B, the training dataset 134 and testing dataset 136 may be applied to the modelling system 106, which may comprise a pre-award module 224, a post-award module 226, and a validation module 218. Such a modelling system 106 may comprise one or more machine learning models configured to, whether individually and/or collectively, generate a predictive trial output relating to, for instance, clinical trial site selection, patient recruitment, and other initiation and/or activation procedures relevant to clinical trial start-up. In at least one embodiment, such a modelling system 106 may be developed and/or provided via a data intelligence platform, such as that provided by Databricks Inc., although other similar platforms providing, for instance, the functionality of the modelling system 106 described herein are contemplated.

As previously discussed, such a pre-award module 224 may be configured to generate a predictive trial output 228 related to the bid and planning phase of a clinical trial—i.e., those phases of a clinical trial occurring before a clinical trial is approved by and/or receives funds from a regulatory agency. Such a post-award module 226, meanwhile, may be configured to generate predictive trial outputs 228 related to the phases of a trial after a study is awarded, approved, and/or granted. Hence, the post-award module 226 may be configured to generate its predictive trial output 228 according to real-time data collection and the present status of a clinical trial as the same proceeds through the various processes thereof. As such, the post-award module 226 may be configured to reforecast a predictive trial output 228 previously generated by the pre-award module 224 once actual data relating to the start-up procedures of a clinical trial are received. The validation module 218 of the modelling system 106, meanwhile, may be configured to train and tune the various models of the pre-award module 224 and/or the post-award module 226.

In at least one embodiment, such a pre-award module 224 may comprise a several discrete models directed to different outputs, as will be discussed in greater detail hereafter. In at least one embodiment, such a post-award module 226 may comprise one or more models configured to predict a sequence of patient counts for each month, such as by using actual, real-time data collected after a trial is awarded and/or activated. For instance, such a post-award module 226 may comprise one or more post-award models configured to generate a predictive trial output 228 in accordance with the disclosure herein, wherein such post-award models may themselves comprise, for instance, sequence-based dep learning models such as, long short-term memory, convolutional neural networks, and a transformer.

As may be seen with continued reference to FIG. 1B, the outputs of the modelling system 106 may be circulated back through the data warehouse 104, such as through an output module 130. Such outputs may subsequently pass to a forecasting system 110, such as through a server 108. Such a server 108 may comprise, without limitation, a centralized computer program and/or process, whether based in hardware or software, configured to provide information to other client computers and/or devices on a network, as understood by those having skill in the art. Such a forecasting system 110 will be discussed in greater detail hereafter.

As may be understood with reference to FIG. 1C, the forecasting system 110 described herein may be accessible and/or operable by one or more user devices 112. Such user devices 112 may comprise, for instance, a laptop, a smartphone, a tablet, or some other similar such device owned and/or operated by a user of the forecasting system 110. Such a user device 112 may comprise, for instance: (a) an electronic processor 140, such as an electronic microprocessor, a microcontroller, or some other similar such component; (b) a memory 148, such as a non-transitory, computer-readable memory; (c) an input/output interface 144, such as some component and/or structure configured to transfer information between the memory 148 and one or more peripheral devices; (d) a human-machine interface 146, such as a touchscreen, a display, a keypad, keyboard, cursor-controlled device, or any other similar such component and/or combination thereof configured to receive data from a user and provide data to a user, whether in a audible, textual, or graphical, or otherwise. Such a user devices 112 may additionally comprise a plurality of additional and/or alternative components, such as electronic processors or memories, application specific integrated circuits, other input devices, other output devices, or otherwise, as may be understood by those having skill in the art.

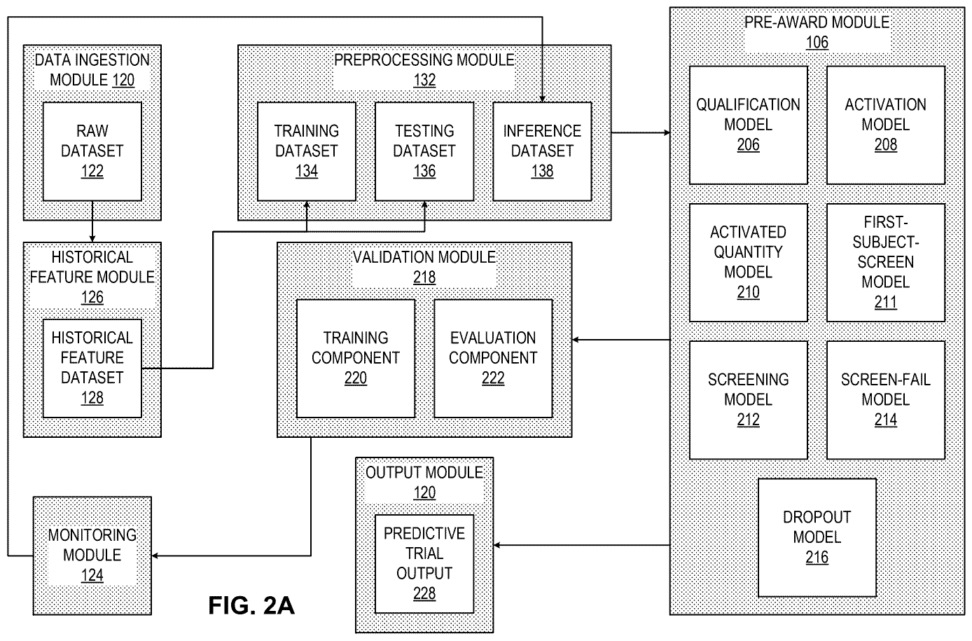

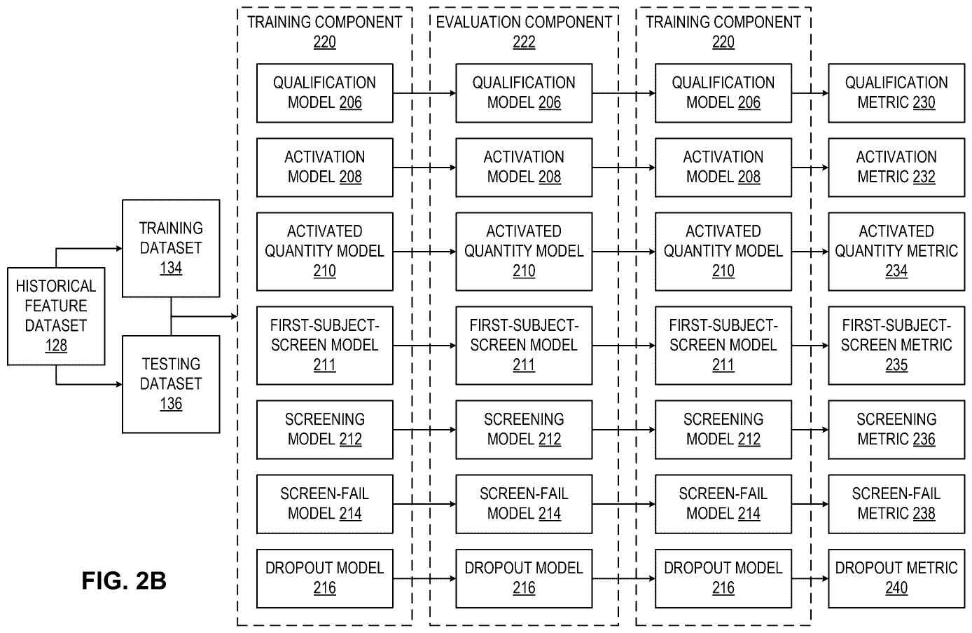

Depicted in FIG. 2A is a diagram depicting data flow between the data warehouse 104 and the pre-award module 224 of the modelling system 106 for the generation of a predictive trial output 228, in accordance with at least one embodiment of the present invention. As previously discussed, the raw dataset 122 generated by the data ingestion module 120 may be transformed into a historical feature dataset 128 by a historical feature module 126, before subsequently being transformed into a training dataset 134 and a testing dataset 136 by a preprocessing module. Such a training dataset 134 and testing dataset 136, as well as an inference dataset 138 generated from data collected by a monitoring module 124 of the data warehouse 104, may be fed into the various models of the pre-award module 224. Such models of the pre-award module 224 may be trained and evaluated by a validation module through, for instance, a training component 220 and an evaluation component 222 thereof. For instance, such a training component 220 may be configured to train and tune the models using hyperparameter tuning, such as through a Bayesian-style HPO with cross-validation. Such a validation module 218 may contribute the generation of an inference dataset 138, such as by passing certain data distribution metrics and/or performance metrics, such as post-qualified metrics, pre-qualified metrics, and pre-feasibility metrics, to a monitoring module 124 of the data warehouse 104. In at least one embodiment, such inference dataset 138 may be batched daily.

More specifically, as may be seen in FIG. 2B, such a pre-award module 224, and particularly the various models thereof, may be trained and evaluated in parallel. And, in at least one embodiment, the target of any one model of the pre-award module 224 may be configured as a feature in a subsequent model. Thus, it may be understood the testing dataset 136 and/or training dataset 134 derived from the historical feature dataset 128 may be fed into the qualification model, activation model 208, activated quantity model 210, first-subject-screen model 211, screening model 212, screen-fail model 214, and dropout model 216 of the pre-award module, for training such models through the training component. Subsequently, each such model may be evaluated according to the evaluation component 222, before undergoing another training session by the training component. As a result, the pre-award module 224 may generate, for example: (a) a qualification metric 230 from the qualification model 206; (b) an activation metric 232 from the activation model 208; (c) an activated quantity metric 234 from the activation model 208; (d) a first-subject-screen metric 235 from the first-subject screen model 211; (e) a screening metric 236 from the screening model 212; (f) a screen-fail metric 238 from the screen-fail model 214; and (g) a dropout metric 240 from the dropout model 216. As may be understood, the above-referenced targets of the various models may comprise features in subsequent models; hence, in at least one embodiment, the qualification metric 230 may be used as a feature in the activation model 208, the activation metric 232 may be used as a feature in the activated quantity model 210, the activated quantity metric 234 may be used as a feature in the screening model 212, the screening metric may be used as a feature in the screen-fail model 214, and the screen-fail model may be used as a feature in the dropout model 216. However, it may be understood alternative arrangements of the models and interplay between the targets and features thereof is envisioned herein. Each of these models will be discussed in greater detail hereafter.

The qualification model 206 of various embodiments of the present invention may be configured to determine when a first site is qualified for a given clinical trial study. In other words, this model is directed to the date at which an initial research site has successfully met all necessary requirements and criteria set by a sponsor of a trial and/or the regulatory authority for the trial. As such, the qualification model 206, in at least one embodiment, may consider data relating to, for instance, obtaining necessary approvals, completing necessary training, meeting infrastructure and equipment requirements, and demonstrating the ability to enroll and manage patients according to the protocol of a given clinical study. Accordingly, it may be understood the qualification metric 230 of at least one embodiment of the present disclosure may comprise a date predicting when a first site will be qualified for a study and/or the number of days until a first site will qualify. In certain embodiments, the qualification metric 230 may be determine according to, for instance, a given country in which a study will be performed.

The activation model 208 of various embodiments of the present invention may be configured to determine when a first site has completed all necessary steps required to initiate patient enrollment and data collection for a trial. Alternatively put, such an activation model 208 may predict when a first site will meet all requirements by a sponsor and/or regulatory authority for the activation of a site, including obtaining necessary approvals, completing training procedures, performing site initiation visits, and otherwise ensuring appropriate readiness for both staff and site infrastructure. As such, the activation metric 232 may, in at least one embodiment, comprise a date when a first site is activated for a given clinical study and/or the number of days between the qualification and activation of given site(s).

Similarly, the activated quantity model 210 of various embodiments of the present invention may be configured to determine a weekly ramp rate indicating the number of sites activated for a given study per week, and per country. Hence, the activated quantity metric 234 may provide a numerical metric indicative of the number of sites that will activate throughout a given period of time.

The first-subject-screen model 211 may be configured to forecast a temporal metric representing when screening procedures for a first subject may begin at a given site or country. The first-subject-screen model 211 may be positioned in a model chain between the activation-related models (such as the activation model 208 and the activated quantity model 210) and the screening model 212. In some examples, the first-subject-screen model 211 may utilize, as inputs, one or more activation-related features derived from the activation metric 232 and/or the activated quantity metric 234, together with historical features describing prior studies, therapeutic areas, countries, and investigators. In various implementations, the model may additionally consider engineered temporal features describing historical intervals between site activation and an initial subject-screening or first-subject-first-visit event, as well as protocol-specific screening window characteristics.

The output of the first-subject-screen model 211 may include a first-subject-screen metric 235 representing a predicted duration between site activation and a first subject-screen event, such as a first-subject-first-visit event. In some examples, the first-subject-screen metric 235 may include a number of days until the first subject is predicted to be screened, whereas in various implementations the first-subject-screen metric 235 may comprise a predicted calendar date generated by combining the modeled duration with the activation metric 232. In response to generation of the first-subject-screen metric 235, the screening model 212 may utilize the first-subject-screen metric 235 as a temporal anchor to determine when a predicted screening rate may begin to apply. Accordingly, the first-subject-screen metric 235 may provide an intermediate milestone that refines downstream screening and enrollment forecasts used to generate the predictive trial output 228.

The screening model 212 of various embodiments of the present invention, meanwhile, may be configured to determine a screening metric 236, such as a screening rate for a given trial. Such a screening rate may comprise the number and/or percentage of individuals who undergo a screening process in relation to the total number of individuals approached and/or considered for participation in a given trial. As such, the screening metric 236 may represent the proportion of individuals who proceed to the screening stage of a trial out a total pool of potential participants. Such a screening metric 236 may consider certain types of data such as, without limitation, an applicable screening window—i.e., the period during which potential participants may be evaluated for eligibility—for a given trial. In at least one embodiment, the screening metric 236 may be configured to provide a range of potential outcomes, such as a best-case scenario and a worst-case scenario, thereby providing additional information relevant to a predictive trial output 228.

The screen-fail model 214 of various embodiments, meanwhile, may be configured to determine a screen-fail metric 238 such as a screen-fail percentage for a given trial. Such a screen-fail percentage may represent the percentage of individuals who undergo a screening process but do not meet the eligibility criteria for participation in a trial. Hence, the screen-fail metric 238 may represent the proportion of individuals who do not pass the screening procedures for a trial and are thus excluded from participation therein.

Similarly, the dropout model 216 of various embodiments may determine a dropout metric 240 such as a forecasted dropout percentage for a trial. Such a dropout percentage may indicate the estimated number of participants who may withdraw or otherwise discontinue their participation in a trial before their completion thereof. As with the screening metric 236, such a dropout metric 240 may be configured to provide a range of potential outcomes, such as a best-case scenario and a worst-case scenario.

As previously discussed, such various models of the pre-award module 224 may comprise, in at least one embodiment, an extreme gradient boosted decision tree model, such as XGBoost, and/or a deep learning model, such as a sequence-based deep learning model. Certain models may utilize one or more transformations or other statistical techniques to generate a better target therefrom. For instance, the qualification model 206, screening model 212, and screen-fail model 214 may each comprise an XGBoost model combined with a Yeo-Johnson transform to account for more flexible skewing and stabilize variance, whereas the dropout model 216 may comprise an XGBoost model utilizing a log(1)p transformation to positively skew the distribution. In contrast, the activation model 208 and the activated quantity model 210 may instead simply comprise an XGBoost. However, it may be understood the use of different models and alternative transformations and/or statistical techniques for each of the models of the pre-award module are envisioned herein. In view thereof, it may be understood the various outputs of the various models of the pre-award module 224 may be collectively used to create a predictive trial output 228 based on the historical feature dataset 128, and the testing dataset 136 and training dataset 134 generated therefrom, as well as an inference dataset 138.

In some examples, the first-subject-screen model 211 includes an extreme gradient boosted decision tree model, such as an XGBoost-based regressor, configured to learn historical intervals between activation and first-subject-screen events. The model architecture may incorporate preprocessing components shared with other models of the pre-award module 224, including categorical encoders, imputers, and one or more transformations applied to skewed cycle-time features. In various implementations, the first-subject-screen model 211 may output a continuous prediction representing an expected number of days between activation and the first subject screen, and this predicted duration may be combined with the activation metric 232 to generate the first-subject-screen metric 235. In some examples, the first-subject-screen model 211 may additionally utilize the activated quantity metric 234 as an upstream feature in scenarios where an expected activation volume for sites within a study or country may influence the timing of subsequent initiation of screening activity.

Returning to FIG. 2A, the evaluation component 222 of the validation module 218 may be configured to evaluate the predictive trial output 228 via one or more evaluation processes. For instance, such an evaluation component 222 may utilize standard error evaluation to determine an overall test median absolute error according to, for instance, historical trial data. Further, such an evaluation component 222 may utilize a baseline evaluation to evaluate, for instance, the median absolute error in view of the historical value for such metric. Further, key performance indicators, such as the frequency with which a model may correctly predict its target within a predetermined error amount, may also be utilized by the evaluation component 222. Such an evaluation component 222 may additionally utilize feature importance processes, such as calculating weight and gain for the models comprising XGBoost, and/or determining SHAP values—i.e., shapely additive explanations—each of which may be configured to indicate how the various features of the historical feature dataset 128, training dataset 134, and/or testing dataset 136 are contributing to the performance of the models.

As previously discussed, the predictive trial output 228 generated by the modelling system 106 of at least one embodiment of the present invention may be transferred to a forecasting system 110, such as through the data warehouse 104 and a server 108 interconnected therewith. There, such a predictive trial output 228 may be configured for display to and interaction with users via one or more graphical user interfaces provided to the user device(s) 112 by the forecasting system 110. In at least one embodiment, such a forecasting system 110 may comprise a software-as-a-service, a web application, or some other similar such system accessible through a web browser, as understood by those having skill in the art.

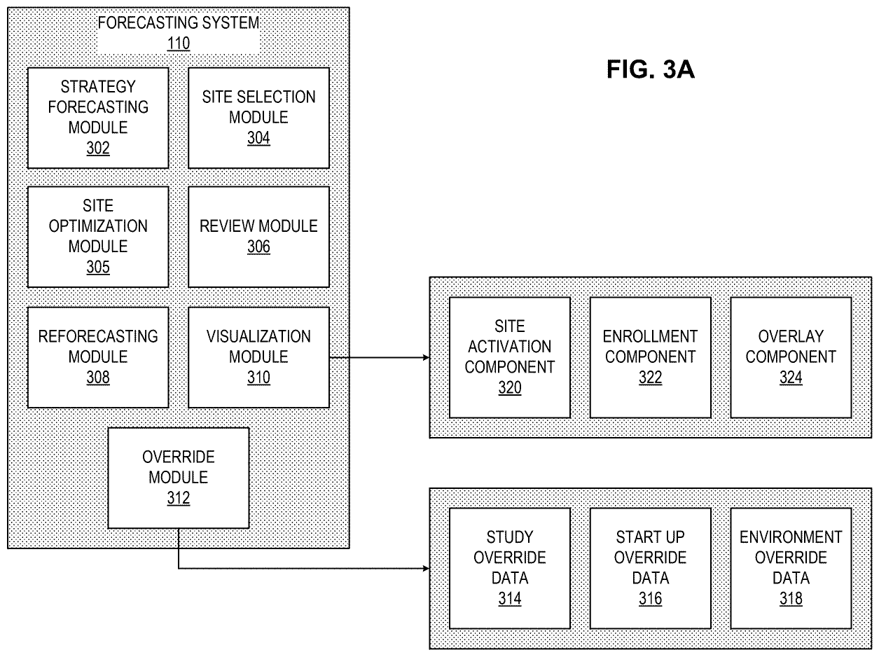

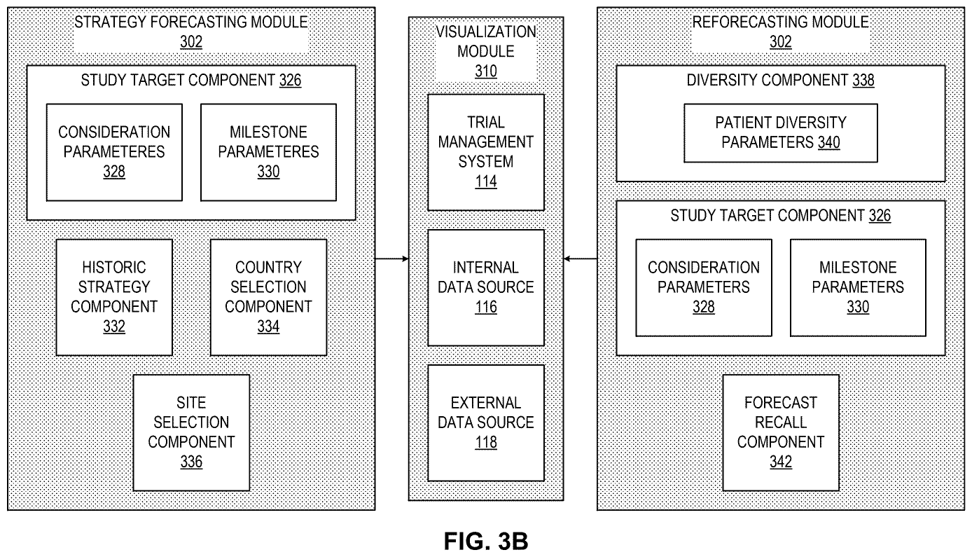

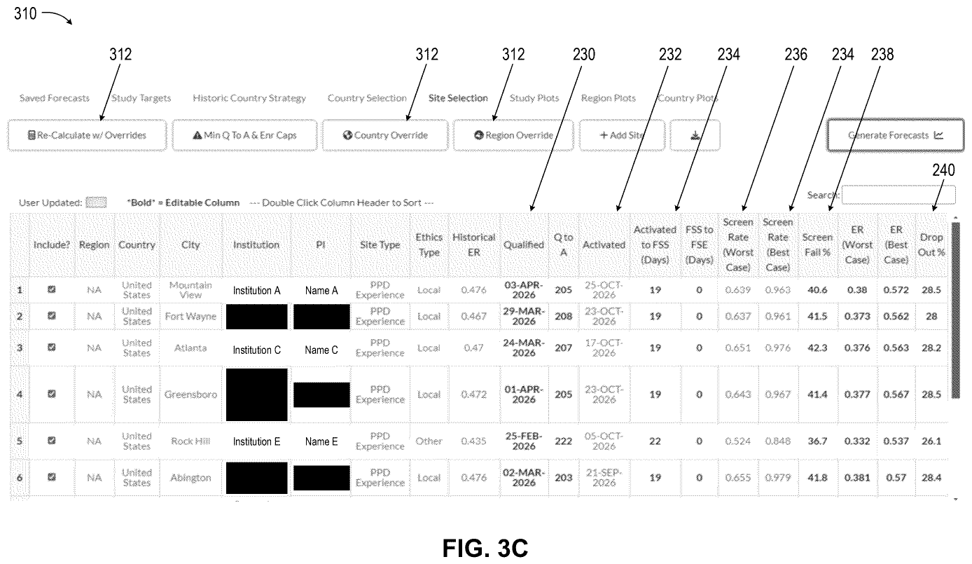

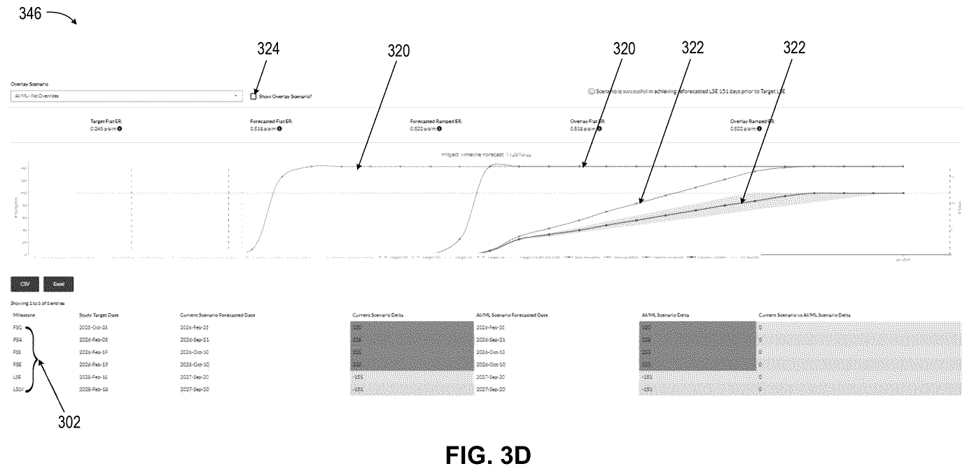

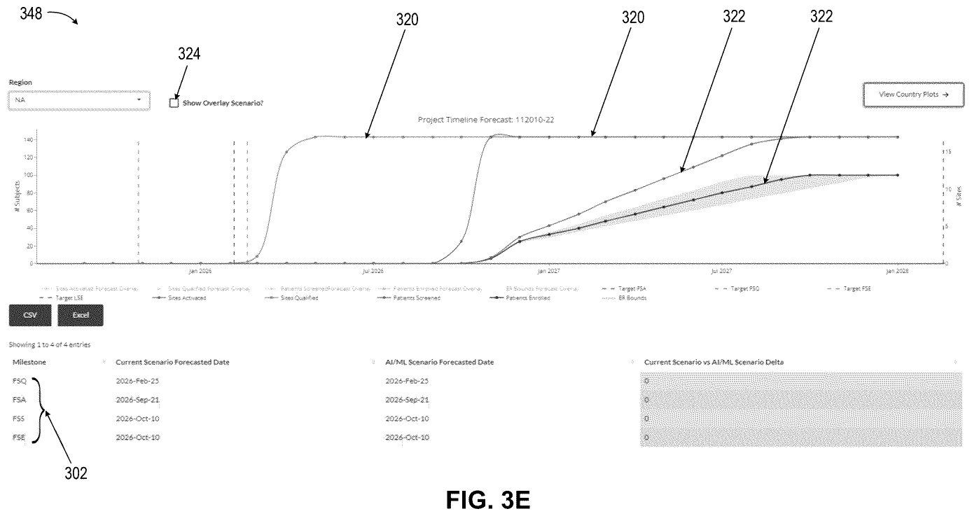

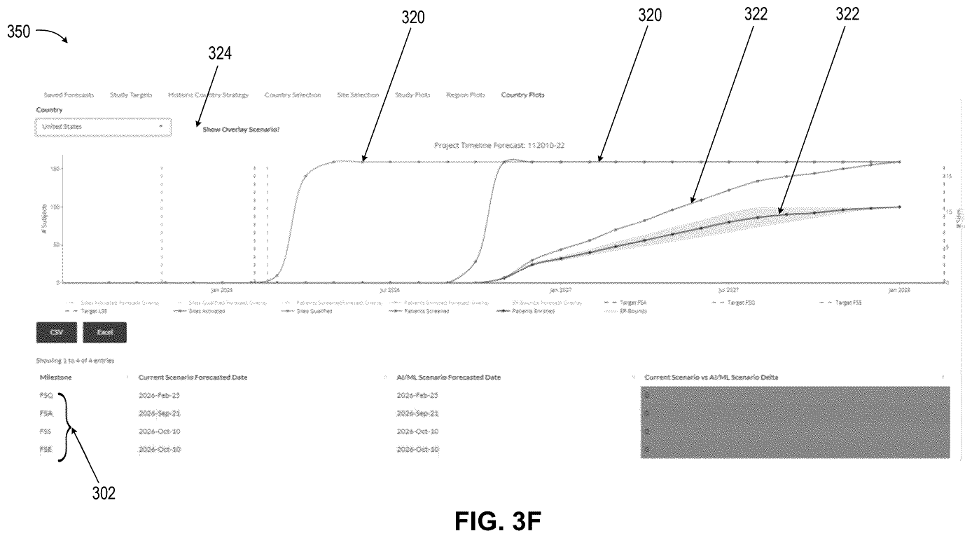

One embodiment of such a forecasting system 110 may be seen with reference to FIG. 3A. As may be seen, such an embodiment of the forecasting system 110 may comprise one or more modules and/or components configured to enable the users thereof to perform the forecasting operations of the trial planning system 10 described herein, including the input of data relevant to a given trial, generating predictive trial output(s) 228 directed to that data, generate visualizations relating thereto, and/or override one or more aspects of the predictive trial output(s) 228 to particularly tailor the same for the specifics of a particular trial. In so doing, it may be understood such a forecasting system 110 may therefore enable a user to develop a comprehensive view of the potential forecasts of a trial at a minute level, such as how start-up and recruitment procedures may be impacted at both a country and individual site level as well as how diversity requirements may dictate the success of trial startup in relation thereto.

For instance, at least one embodiment of a forecasting system 110 in accordance with the present disclosure, such as that depicted in FIGS. 3A and 3B, may comprise a strategy forecasting module 302 and a site selection module 304. Collectively, such a strategy forecasting module 302 and such a site selection module 304 may enable a user to input one or more parameters dictating the needs of a given trial and select one or more preferred sites suiting those needs.

At least one embodiment of such a strategy forecasting module 302, such as the one depicted in FIG. 3B, may comprise study target component 326 through which a user may input one or more consideration parameters 328 and/or milestone parameters 330. Such consideration parameters 328 may comprise study-specific considerations relating to the performance of a clinical trial. For instance, and without limitation, such consideration parameters 328 may comprise a target study date, the number of patients to be involved in a trial, a preferred screening window length, a preferred enrollment period length, the duration for treatment and/or follow-up procedures, and seasonality factors. Such milestone parameters 330, in contrast, may comprise parameters relating to intended milestones for both start-up and enrollment procedures, such as target dates for the completion of certain actions relating thereto. For instance, such milestone parameters may include the target dates for first site qualification, first site activation, last site activation, first subject screened, first subject enrolled, last subject screened, last subject enrolled, and a last subject's last clinical trial visit, as may be understood in view of the disclosure heretofore. In so doing, it may be understood a user of the forecasting system 110 may be enabled to input a variety of parameters dictating how they would like a trial to be performed, thereby enabling a tailored forecast for the predictive trial output 228.

Such a site selection module 304, meanwhile, may comprise a catalog, list, database, or some other similar data structure comprising an overview of a plurality of clinical trial sites, such as those known through the system—e.g., those sites owned and/or operated by the owner and/or operator of the forecasting system 110 and/or trial planning system 10—or those sites identified within the raw dataset 122 received from the data source(s) 102. In at least one embodiment, such a site selection module 304 may include the name of a given clinical trial site, along with a plurality of data relating thereto, such as, without limitation, geographic information, such as the site's country and city, and institutional information, such as a site's institution type, its principal investigator—i.e., the healthcare professional who may be involved in performing the trial—, and other institutional information relating to the site's ownership and ethical oversight. In at least one embodiment, such a site selection module 304 may additionally comprise one or more algorithms, such as an artificial intelligence algorithm, configured to generate a list of sites most suited to the consideration parameters 328 and/or milestone parameters 330 input by the user through the strategy forecasting module 302. Accordingly, such a site selection module 304 may enable a user to easily identify potential sites for a given trial, and, through the trial planning system 10 described herein, assess the ability of such potential sites to meet the intended parameters under which a trial will operate. In at least one embodiment, such a site selection module 304 may be configured in connection with the strategy forecasting module 302, such via a site selection component 336 and/or country selection component 334 thereof, thereby enabling a user to specify specific sites and/or countries for a given forecast.

In various implementations, the forecasting system 110 includes a site optimization module 305 configured to compute an optimized selection and configuration of clinical trial sites according to predicted site-performance metrics, such as the predictive trial output 228 generated by the pre-award module 224 and/or one or more inputs received from a user through a user device 112. The user device 112 may transmit site-optimization parameters to the forecasting system 110 through the input/output interface 144 and may generate a graphical user interface including corresponding controls, menus, and interactive elements, which may be presented to the user through the human-machine interface 146. In some examples, the site optimization module 305 may receive consideration parameters 328 and milestone parameters 330 provided through the strategy forecasting module 302, as well as other site-optimization parameters received from the user device 112 and generate an optimized configuration of clinical trial sites that reflects the user's constraints, preferences, and operational objectives.

In various implementations, the site optimization module 305 may utilize as inputs the qualification metric 230, activation metric 232, activated quantity metric 234, screening metric 236, screen-fail metric 238, and dropout metric 240 generated by the pre-award module 224, as well as the first-subject-screen metric 235 generated by the first-subject-screen model 211. In some examples, the site optimization module 305 may additionally receive one or more user-defined parameters transmitted from the user device 112 through the input/output interface 144, where such parameters may be entered, selected, or adjusted by the user through the human-machine interface 146. These user-defined parameters may include hard criteria that define minimum or maximum allowable conditions for a trial, soft criteria that define preferences or weighted desirability measures, site-specific or country-specific diversity requirements, regional or country constraints, enrollment targets, and timeline objectives associated with one or more milestone parameters 330. In various implementations, the site optimization module 305 may treat the above inputs collectively as an input parameter set used to configure a site-optimization problem for a given trial.

In some examples, the site optimization module 305 may generate outputs comprising an optimized set of clinical trial sites selected from a broader pool of candidate sites, an optimized allocation of sites across countries or regions, an optimized activation schedule for such sites, an optimized enrollment projection for the study period, and one or more diagnostic indicators describing how the resulting optimized configuration satisfies the various hard criteria and approximates satisfaction of the soft criteria. In various implementations, the site optimization module 305 may return these optimized results to other components of the forecasting system 110, including the strategy forecasting module 302, the reforecasting module 308, and the visualization module 310, thereby permitting updated plots, tables, or scenario comparisons to be displayed to a user through the human-machine interface 146 of the user device 112.

In various implementations, the site optimization module 305 applies an optimization algorithm to identify a feasible and preferably high-performing site configuration that balances the site-performance predictions of the pre-award module 224 with one or more user-specified constraints and preferences. In some examples, the optimization algorithm considers the site-performance metrics collectively as feature inputs describing potential throughput, timeline feasibility, and patient-recruitment characteristics at each candidate site. In various implementations, the optimization algorithm considers multiple objective styles. In some examples, the optimization algorithm treats the site-optimization problem as a multi-objective problem in which criteria relating to timeline feasibility, enrollment strength, diversity coverage, and soft-criteria satisfaction are combined into a weighted objective. In additional examples, the optimization algorithm treats the site-optimization problem as a flexible objective framework in which different objectives may be prioritized individually, sequentially, or in combination, according to one or more parameters provided by the user through the human-machine interface 146. In various implementations, the optimization algorithm computes one or more optimized solutions and selects an optimized output configuration based on one or more configurable performance thresholds.

In some examples, the site optimization module 305 applies a mixed-integer linear optimization (MILO) technique to compute an optimized site configuration from among a pool of candidate sites. In such examples, the optimization problem may include binary decision variables representing whether a site is selected, integer decision variables representing a number of sites to be activated in a given country or region, and continuous decision variables representing enrollment-related or timeline-related quantities. In various implementations, the MILO formulation may include linear constraints relating to minimum and maximum enrollment expectations, maximum screening capacity, maximum dropout tolerance, required diversity distributions, and country-specific limitations, each of which may be derived from the site-performance metrics generated by the pre-award module 224 or from user inputs entered through the human-machine interface 146 and transmitted through the input/output interface 144. In some examples, the MILO formulation may include an objective function configured to balance early activation, strong enrollment performance, desired diversity levels, reduced operational risk, or increased site-performance strength, as may be specified through the user device 112. In various implementations, the site optimization module 305 uses the MILO solution to produce a corresponding optimized set of clinical trial sites, optimized activation timing, and optimized projected enrollment contributions in a manner that reflects the constraints provided to the site optimization module 305.

In various implementations, a user may adjust site-optimization parameters, criteria weights, or scenario assumptions through the graphical user interface output via the human-machine interface 146, and such adjustments may be communicated to the forecasting system 110 through the input/output interface 144. In some examples, the site optimization module 305 recomputes an optimized site configuration in response to such adjustments, thereby allowing iterative refinement of the site-selection scenario. In various implementations, the optimization results are displayed on the user device 112 as updated tables, updated country-allocation summaries, updated activation-timing projections, or updated enrollment forecasts. In some examples, the user may use the reforecasting module 308 to compare the outputs generated from successive optimization runs to evaluate tradeoffs among enrollment, diversity, and timeline objectives.

In various implementations, the site optimization module 305 receives predictive site-performance metrics from the pre-award module 224 and one or more user-provided optimization parameters from the user device 112, compute an optimized configuration in accordance with generic or MILO-based optimization techniques, and forward the resulting optimized outputs to the visualization module 310. In some examples, the visualization module 310 may generate corresponding plots illustrating the optimized activation curve and enrollment curve, with such plots being updated in response to subsequent optimization iterations or adjustments to the underlying inputs. Thus, the site optimization module 305 may provide a configurable, data-driven mechanism for constructing site-selection and planning scenarios that incorporate predicted performance, user-defined criteria, diversity parameters, country-level constraints, enrollment targets, and timeline objectives for a given clinical study.

Returning to FIG. 3A, it may be seen at least one embodiment of the forecasting system 110 described herein may additionally comprise a review module 306. Such a review module 306 may be configured to enable one or more users to review, for instance, selected sites and/or countries for a given trial. For instance, such a review module 306 may be configured to generate one or more trend plots, to be displayed to a user via the visualization module 310 of the forecasting system 110. Such trend plots may be configured to display, for instance, trend data relating to, without limitation, the number and nature of trials performed at such sites and/or within such countries based on, for instance, the raw dataset 122 received from the data source(s) 102. Hence, it may be understood such trend plots may incorporate data from both internal data source(s) 116 and external data source(s) 118, thereby providing a comprehensive overview of how other trials, such as those having similar parameters to those consideration parameters 328 and/or milestone parameters 330 input by a user, have been conducted at a country and/or site level. In at least one embodiment, such a review module 306 may be configured for use by the strategy forecasting module 302, such as through a historic strategy component 332 thereof, to enable a user to select one or more trials for consideration in connection with a predictive trial output 228 for a given trial.

Further, at least one embodiment of such a forecasting system 110 may additionally include a reforecasting module 308. Such a reforecasting module 308 may be configured to save and recall previously conducted forecasts, such as one or more predictive trial output(s) 228, and subsequently edit those forecasts and/or compare such forecasts with one or more alternative forecasts. As may be understood, at least one embodiment of such a reforecasting module 308, such as the one depicted in FIG. 3B, may be configured to utilize the study target component 326 discussed heretofore, such as the consideration parameters 328 and/or the milestone parameters 330 input into the forecasting system 110 therefrom, to adjust the various parameters and timelines used in connection with a predictive trial output 228 to effectuate such edit(s) and/or comparison(s). Similarly, such a predictive trial output 228 may also be configured for editing and/or adjusting previous forecast using the site selection module 304 discussed heretofore, such as to select alternative sites and/or countries for conducting a clinical trial. For example, in at least one embodiment, such a reforecasting module 308 may be configured to generate at least one additional predictive trial output 228 according to at least one previous predictive trial output 228, such as by altering consideration parameters 328, milestone parameters 330, selected sites, and/or selected countries, and compare the same with such previous predictive trial output 228, thereby enabling a user to obtain different forecasts based on different features or parameters of a clinical trial.

In various implementations, the reforecasting module 308 includes a forecast recall component 342 configured to retrieve previously stored predictive trial outputs. The forecast recall component 342 may access one or more stored forecast records in response to a user input received via the human-machine interface 146 of the user device 112 and may transmit the recalled forecast to the visualization module 310, the site optimization module 305, and/or the override module 312 for further analysis, comparison, or modification.

Further, the reforecasting module 308 of at least one embodiment may additionally include a diversity component 338, through which a user may input one or more patient diversity parameters 340 to be used in connection with a clinical trial. Such patient diversity parameters 340 may include, for instance, sex diversity metrics, age diversity metrics, Hispanic diversity metrics, race allocation metrics, gender allocation metrics, and other similar such diversity-based metrics that may be applicable to a given clinical trial. Such patient diversity parameters 340 may be utilized to increase the insight of a given study forecast based on the predictive trial output 228 connected therewith. For instance, such patient diversity parameters 340 may, in at least one embodiment, be configured in connection with the site selection module 304 to indicate sites and/or countries most suited to meet the input diversity requirements based on, for instance, the raw dataset 122 and/or the historical feature dataset 128.

Returning to FIG. 3A, at least one embodiment of the forecasting system 110 of the present invention may additionally comprise an override module 312. Such an override module 312 may be configured to enable one or more override inputs in relation to the predictive trial output(s) 228 generate by the modelling system 106. Such an override module 312 may receive from a user, for instance, study override data 314, start up override data 316, and/or enrollment override data 318 through which a user may override one or more aspects of the modelling system 106. In so doing, a user may be enabled to specifically tailor certain aspects of a predictive trial output 228. For instance, the override module 312 may enable a user to input one or more override parameters including, without limitation, a specific enrollment rate, a specific number of patients who may be registered and screened for a trial, a specific country, or a specific type of site experienced in a specific therapeutic area. In so doing, the override module 312 may enable a user to, in at least one embodiment, dictate how the training dataset 134 and/or testing dataset 136 is formed from the historical feature dataset 128, thereby impacting how the modelling system 106 generates a predictive trial output 228.

With continued reference to FIGS. 3A and 3B, at least one embodiment of the forecasting system 110 described herein may comprise a visualization module 310. Such a visualization module 310 may be configured to generate one or more graphic interfaces depicting, for instance, a predictive trial output 228 generated by the modelling system 106, such as in the form of a table, chart, or other similar graphic interface. For instance, as depicted in FIG. 3C, such a visualization module 310 may display a predictive trial output 228 in the form of a table, indicating, for instance, various sites and other relevant information relating thereto, and the qualification metric 230, activation metric 232, activated quantity metric 234, screening metric 236, screen-fail metric 238, and/or dropout metric 240 associated therewith. Further, as may be seen with continued reference to FIG. 3C, such a visualization module 310 may enable a user to recalculate the predictive trial output 228 displayed thereon with one or more selectable override features, through which a user may input various override parameters and subsequently recalculate an additional predictive trial output 228 in connection therewith.

Likewise, the visualization module of at least one embodiment may also be configured to receive the inputs of, for instance, the strategy forecasting module 302 and/or the reforecasting module 308 and apply the same in connection with the predictive trial output(s) 228 received from the modelling system 106 to generate one or more visualizations relating thereto. Such visualizations generated by the visualization module 310 may comprise, for instance, one or more plots, such as a study plot 346, a region plot 348, and/or a country plot 350 depicting a given forecast at varying levels of depth. In connection therewith, each such visualization of the visualization module 310 may include one or more curves indicating one or more aspects of the predictive trial output(s) 228. For instance, a site activation component 320 may be utilized to generate a site activation curve depicting, for instance, the qualification metrics 230, activation metrics 232, and/or activated quantity metrics 234 of a predictive trial output 228 as applied in relation to a user's inputs. An enrollment component 322 of the visualization module 310, meanwhile, may be used to generate an enrollment curve depicting, for instance, the screening metrics 236, screen-fail metrics 238, and/or dropout metrics 240 of a predictive trial output 228 in view of the user's inputs. Furthermore, an overlay component 324 may be configured to utilize the reforecasting module 308 to overlay plots and/or curves associated with two or more predictive trial outputs 228, such as those generated in connection with different consideration parameters 328, milestone parameters 330, sites, countries, and/or patient diversity parameters 340, thereby enabling a user to visualize how any one decision may impact a given forecast.

For instance, embodiments of such a study plot 346, region plot 348, and country plot 350 may be seen in FIGS. 3D-3F, respectively. There, it may be seen the visualization module 310 may display information relevant to the strategy forecasting module 302, such as the consideration parameters 328 and/or milestone parameters 330 input therethrough. Further, the site activation component 320 may generate at least one site activation curve on the relevant plot, whereas the enrollment component 322 may generate at least one enrollment curve on the relevant plot. Further, an overlay component 324 may be selected to generate at least one overlay plot in connection with the relevant plot.

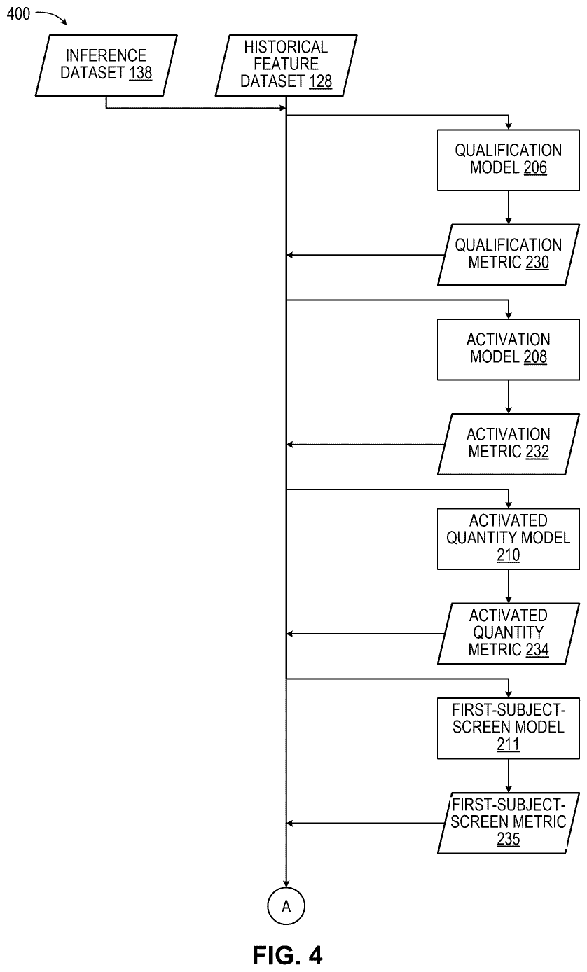

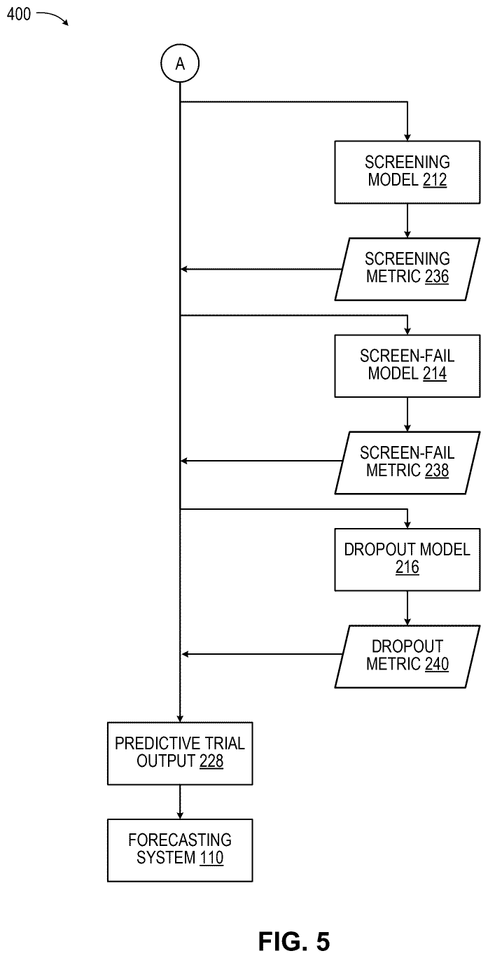

FIGS. 4 and 5 illustrate a block diagram 400 depicting data flow between components of the system 10 during execution of a process for generating, updating, and/or optimizing clinical-trial forecasts using historical data, predictive models, user inputs, and/or real-world operational data. The block diagram 400 depicts two related modelling processes: a pre-award modelling process in which only historical data is available for forecasting purposes, and a post-award modelling process in which real-world performance data from an active clinical study may supplement or refine the information used by the predictive models of the pre-award module 224. The following discussion describes each example process in turn.

The block diagram 400 may illustrate a pre-award process in which the modelling system 106 receives a historical feature dataset 128 generated by the data warehouse 104 from historical data stored in one or more data sources. In such implementations, the historical feature dataset 128 may represent the primary feature set used by each predictive model of the pre-award module 224. During the pre-award process, real-world operational data from a live study may not exist, and the inference dataset 138 may therefore remain unpopulated or unused for forecasting purposes.

Relevant portions of the historical feature dataset 128 may be provided to the qualification model 206. In some examples, the qualification model 206 receives the historical feature dataset 128 as inputs and generate a qualification metric 230 indicating a predicted time period or date at which a first site may achieve qualification for a clinical study. The qualification metric 230 may be stored, processed, and/or forwarded to subsequent models of the pre-award module 224 for use as an input.

The historical feature dataset 128 and/or the qualification metric 230 may be provided to the activation model 208 as input. In some examples, the activation model 208 may generate an activation metric 232 indicating a predicted time period or date at which a first site may reach activation readiness. The activation metric 232 may be output to block diagram 400 and may be utilized by one or more subsequent models of the pre-award module 224.

The historical feature dataset 128, the qualification metric 230, and/or the activation metric 232 may be provided to the activated quantity model 210. In some examples, the activated quantity model 210 may generate an activated quantity metric 234 indicating a predicted number of sites that may activate for a given study within a specified time period. The activated quantity metric 234 may be transmitted to subsequent models of the pre-award module 224.

The historical feature dataset 128, the qualification metric 230, the activation metric 232, and/or the activated quantity metric 234 may be provided to the first-subject-screen model 211. In some examples, the first-subject-screen model 211 may generate a first-subject-screen metric 235 representing a predicted time period or date at which a first subject may be screened for the clinical study. The first-subject-screen metric 235 may be provided to subsequent models for additional processing.

The historical feature dataset 128, the qualification metric 230, the activation metric 232, the activated quantity metric 234, and/or the first-subject-screen metric 235 may be provided to the screening model 212. In some examples, the screening model 212 may generate a screening metric 236 representing a predicted screening rate or screening performance level for the clinical study. The screening metric 236 may be forwarded to subsequent models of the pre-award module 224.

The historical feature dataset 128, the qualification metric 230, the activation metric 232, the activated quantity metric 234, the first-subject-screen metric 235, and/or the screening metric 236 may be provided to the screen-fail model 214. In some examples, the screen-fail model 214 may generate a screen-fail metric 238 indicating a predicted percentage of screened subjects who may not meet eligibility requirements for participation in the study. The screen-fail metric 238 may be provided as an input to the dropout model 216.

The historical feature dataset 128, the qualification metric 230, the activation metric 232, the activated quantity metric 234, the first-subject-screen metric 235, the screening metric 236, and/or the screen-fail metric 238 may be provided to the dropout model 216. In some examples, the dropout model 216 may generate a dropout metric 240 indicating a predicted percentage of subjects who may discontinue participation before completion of the study.

In various implementations, one or more of the historical feature dataset 128, the qualification metric 230, the activation metric 232, the activated quantity metric 234, the first-subject-screen metric 235, the screening metric 236, the screen-fail metric 238, and/or the dropout metric 240 may collectively be used to generate a predictive trial output 228 for use in the forecasting system 110. The predictive trial output 228 generated during the pre-award process may represent a fully model-driven forecast based on historical features and the predictive relationships encoded within the models of the pre-award module 224.

The block diagram 400 further illustrates a related post-award process in which the modelling system 106 receives both the historical feature dataset 128 and real-world performance data captured during the active execution of a clinical study. In various implementations, the monitoring module 124 may receive real-world operational data relating to site performance, patient screening, patient enrollment, and/or subject dropout events. The monitoring module 124 may transmit such performance data to the data warehouse 104, which may generate or update an inference dataset 138. In some examples, the inference dataset 138 may be batched or refreshed periodically and may supplement or refine the feature information used by one or more predictive models of the pre-award module 224. In certain implementations, the inference dataset 138 may replace one or more previously predicted model inputs, thereby allowing the chained models to operate on observed real-world data rather than solely on pre-award predictions.

In various implementations, relevant portions of the historical feature dataset 128, the inference dataset 138, and/or one or more metrics derived from the monitoring module 124 are provided to the qualification model 206, activation model 208, activated quantity model 210, first-subject-screen model 211, screening model 212, screen-fail model 214, and/or dropout model 216, in the same chained sequence described for the pre-award process. In some examples, when the inference dataset 138 contains real-world operational data corresponding to a target predicted by a downstream model in the chain, the corresponding predicted metric may be bypassed, replaced, or updated using such real-world data. In this manner, the chained models may generate updated qualification metrics 230, activation metrics 232, activated quantity metrics 234, first-subject-screen metrics 235, screening metrics 236, screen-fail metrics 238, and/or dropout metrics 240 that incorporate both historical information and observed trial performance. In various implementations, such updated metrics may reflect the changing operational conditions of the study and may therefore support rolling or continuous forecast updates.

In various implementations, one or more of the historical feature dataset 128, the inference dataset 138, the qualification metric 230, the activation metric 232, the activated quantity metric 234, the first-subject-screen metric 235, the screening metric 236, the screen-fail metric 238, and/or the dropout metric 240 are aggregated to generate an updated predictive trial output 228. In some examples, the updated predictive trial output 228 may be transmitted from the modelling system 106 to the forecasting system 110 for generation of updated visualizations, scenario comparisons, and/or optimization operations performed by the site optimization module 305. In various implementations, the post-award process depicted in block diagram 400 may thereby represent a data-augmented update to the initial pre-award forecast, allowing responsive adjustments throughout the lifecycle of the clinical study.

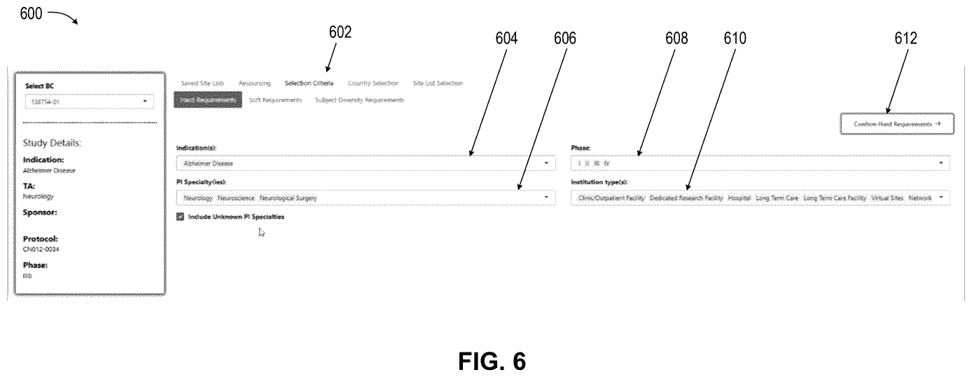

FIGS. 6-9 and 11-17 are diagrams illustrating aspects of a graphical user interface 600 generated by the site optimization module 305, according to some examples. The graphical user interface 600 may be generated by the site optimization module 305 in response to site-selection operations initiated through the user device 112. In various implementations, the site optimization module 305 may produce a user-interface configuration describing layout, fields, and interaction states for a site-selection workflow, and the visualization module 310 may render this configuration and output the rendered interface to the human-machine interface 146 for display to a strategist. Through this rendering process, the strategist may view and interact with the screen elements that define the initial phase of the site-selection workflow.

FIG. 6 illustrates a hard requirements screen through which mandatory site-selection parameters are specified. The graphical user interface 600 may present a selection criteria element 602, an indication element 604, an investigator specialty field 606, a study phase field 608, an institution type field 610, and a confirm hard requirements element 612. Each field or element may appear as a clickable tab, button, drop-down menu, fillable field, or related user-interface control according to the configuration produced by the site optimization module 305.

In various implementations, the strategist accesses this hard requirements screen in response to selecting a control such as “New Site List,” which may cause the site optimization module 305 to present the setup sequence for defining the required selection criteria. At this stage, the FSS team has no activity, as its participation may begin later during a country-team review phase. The strategist may first specify the indication through the indication element 604 and may then specify investigator expertise through the investigator specialty field 606. In some examples, the investigator specialty field 606 may provide an option allowing unknown specialties to be treated as acceptable, such that a site with relevant therapeutic experience but without a recognized principal investigator specialty remains within the candidate pool. The strategist may additionally select a study phase through the study phase field 608 and may select an institution type through the institution type field 610. These selections may function as hard criteria, such that any site failing to satisfy at least one of the values specified on this screen is not passed forward into the candidate site list.

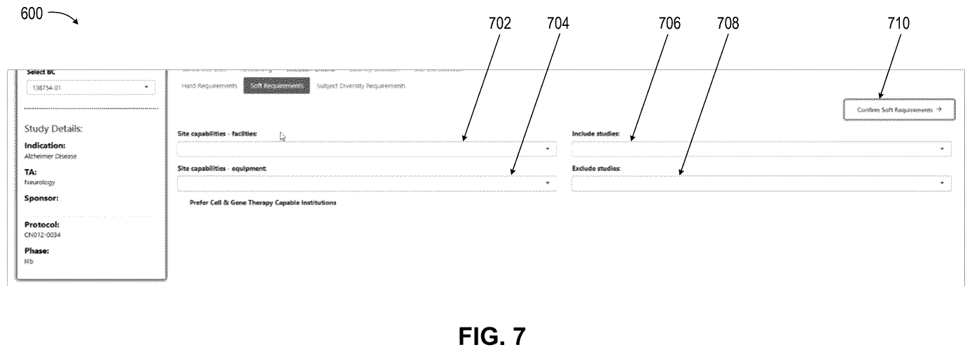

When the strategist activates the confirm hard requirements element 612, the values selected on the hard requirements screen may be finalized or saved for the current workflow instance. In response to this activation, the site optimization module 305 may transition the strategist from the hard requirements screen of FIG. 6 to a soft requirements screen illustrated in FIG. 7, where optional preferences may be applied.

In various implementations, FIG. 7 illustrates a soft requirements screen presented in response to confirmation of the hard criteria specified on the screen of FIG. 6. Once the site optimization module 305 receives the finalized hard-criteria selections, the visualization module 310 may render a subsequent graphical user interface representing optional, non-exclusionary parameters. This soft requirements screen may allow a strategist to refine the site-selection scenario by identifying attributes that strengthen the desirability of a site without disqualifying a site that lacks such attributes.

The graphical user interface of FIG. 7 may present a site capabilities facilities field 702, a site capabilities equipment field 704, an include studies field 706, an exclude studies field 708, and a confirm soft requirements element 710. Each field may appear as a fillable field, drop-down selector, or data grid populated by the site optimization module 305 based on records accessible through the forecasting system 110. The strategist may review or select soft attributes in these fields without modifying the underlying eligibility rules defined by the hard requirements.

In some examples, the include studies field 706 and exclude studies field 708 may provide visibility into study-status information for trials with related therapeutic or operational characteristics. When a strategist is working on a competitive or extension study and possesses a business code corresponding to a related trial, the strategist may observe the related business codes displayed in the include and exclude columns as advisory data. The site capabilities facilities field 702 and site capabilities equipment field 704 may present facilities and equipment characteristics associated with a site. When such information is unavailable for a given site, the associated field may remain blank while the site remains eligible for consideration.

In other examples not illustrated in FIG. 7, additional soft attributes, such as subject-diversity information or other optional characteristics, may appear once the hard criteria are confirmed. Such attributes may not operate as filtering constraints on the candidate site pool for this stage of the workflow. Instead, the parameters selected or reviewed on the soft requirements screen may function as preference-oriented guidance that enhances precision without reducing the eligible site pool.

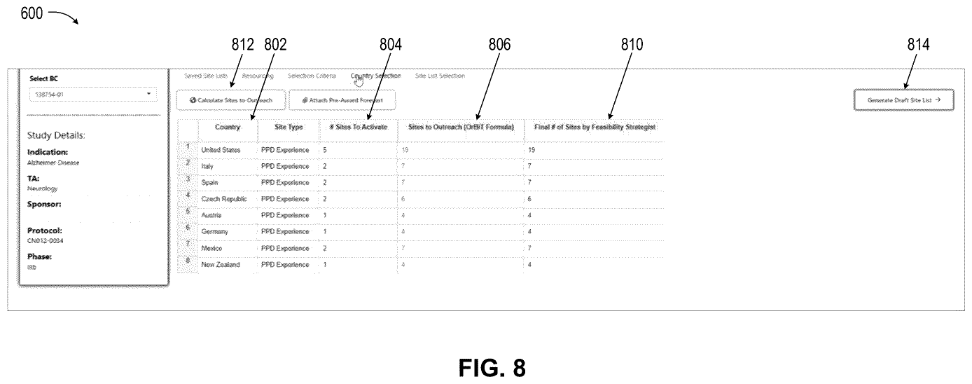

When the strategist activates the confirm soft requirements element 710, the soft-attribute selections associated with the displayed fields may be stored by the site optimization module 305 for the current workflow instance. In response, the site optimization module 305 may transition the strategist to the country selection screen illustrated in FIG. 8, where subsequent geographic and regional selections may be applied.

In various implementations, FIG. 8 illustrates a country selection screen presented in response to confirmation of the soft requirements specified on the screen of FIG. 7. Once the site optimization module 305 stores the strategist's soft-attribute selections, the visualization module 310 may render a graphical user interface through which geographic allocations and country-level site targets are entered. This country selection screen may allow a strategist to define the countries to be evaluated for the study and to assign initial site-count projections that guide downstream selection and optimization logic.

The graphical user interface of FIG. 8 may present a countries column 802, a number of sites to activate column 804, a sites to outreach column 806, and a final number of sites by feasibility strategist column 810. Each column may provide editable rows corresponding to specific countries selected for consideration. The strategist may populate the countries column 802 to identify the geographic scope of the planned study. The strategist may then enter projected values for the number of sites to activate in the number of sites to activate column 804 and projected values for the number of sites to outreach in the sites to outreach column 806. A calculate sites to outreach element 812 may be provided as a selectable control that, in some examples, triggers a computation of recommended outreach counts based on the projected activations and the criteria previously defined on the hard and soft requirements screens. After this computation is presented, the strategist may edit or finalize the outreach values as needed.

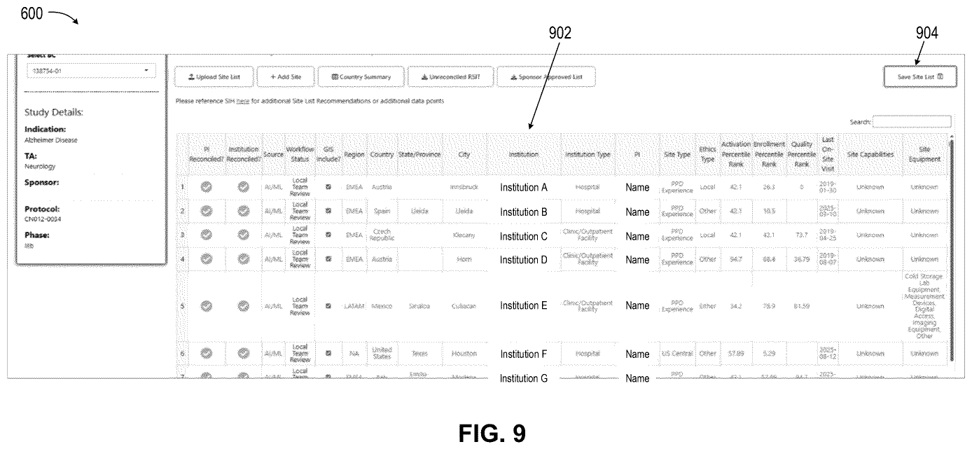

The final number of sites by feasibility strategist column 810 may permit the strategist to enter a finalized target number of sites per country. This final value may reflect the strategist's professional judgment and may serve as a study-level goal used by the site optimization module 305 during subsequent site-search and site-ranking operations. In various implementations, once the strategist has entered or refined the values in the editable columns, the strategist may activate the generate draft site list element 814. Activation of this element may store the country-level selections and may transition the workflow to the initial list of sites screen illustrated in FIG. 9, where the system presents a draft set of candidate sites consistent with the country selections and projections specified on the country selection screen.

In various implementations, FIG. 9 illustrates an initial list of sites screen presented in response to activation of the generate draft site list element 814 on the country selection screen of FIG. 8. In response to that activation, the site optimization module 305 may generate an initial set of candidate sites according to the previously defined hard requirements, soft requirements, and country-level parameters. The visualization module 310 may render the resulting dataset and output the rendered interface to the human-machine interface 146, presenting the strategist with a preliminary table of institutions for further review.

The graphical user interface of FIG. 9 may present an institutions list 902 structured as a table populated with a variety of site-level attributes. In some examples, the institutions list 902 may present columns such as PI reconciled, institution reconciled, source, workflow status, GIS include, region, country, state/province, city, institution, institution type, principal investigator, site type, ethics type, activation percentile rank, enrollment percentile rank, quality percentile rank, on-site visit indication, site capabilities data, and site equipment data. These columns may display values retrieved from internal records, external sources, or previously reconciled information.

In various implementations, when the strategist activates the save site list element 904 and saves the draft list as active, such activation may function as a trigger to notify downstream teams that a preliminary set of sites has been generated. At this stage, the draft site list may not yet be complete, as sponsor-recommended sites or externally sourced sites may not yet have been added. The notification transmitted in response to activation of the save site list element 904 may help feasibility strategists anticipate upcoming assignments and prepare for their involvement in the site review process.

The draft site list may then be refined by the strategist. In some examples, the strategist may add sponsor-recommended sites, upload additional sites not present in the database, or address outstanding records requiring reconciliation. The strategist may download unreconciled site information and transmit those records to internal data-management personnel for evaluation and creation of any missing information. Additional sites may be incorporated into an internal site-information record used to store site attributes for reconciliation and upload into the site selection module. Once the internal records have been updated, the strategist may upload the updated site-information file into the site selection module, perform de-duplication, conduct quality-control review, and apply a local team review workflow status. In response, the list may become visible to local teams, and a notification may be transmitted after the strategist saves the updated list as active. In various implementations, any newly added site may require a valid registry identifier to be successfully stored and used within the trial-planning system.

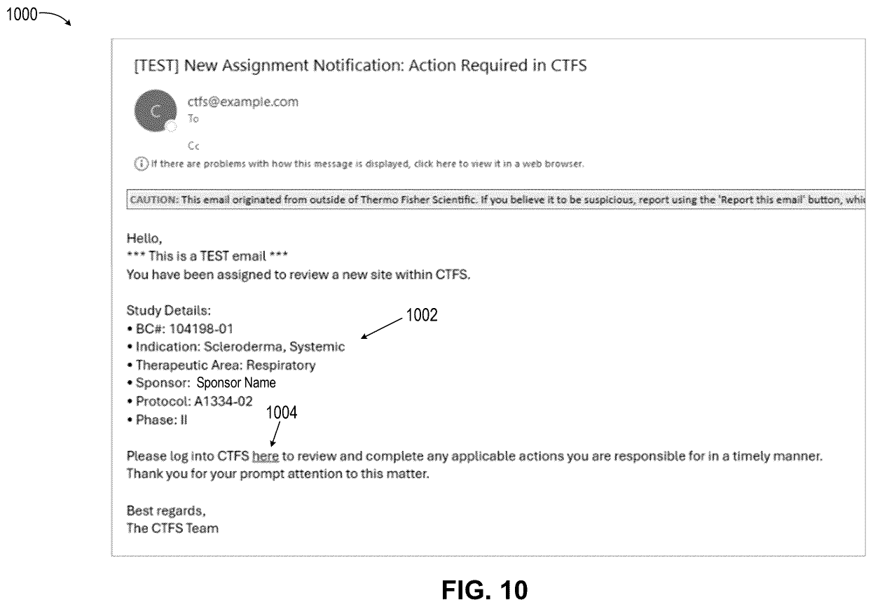

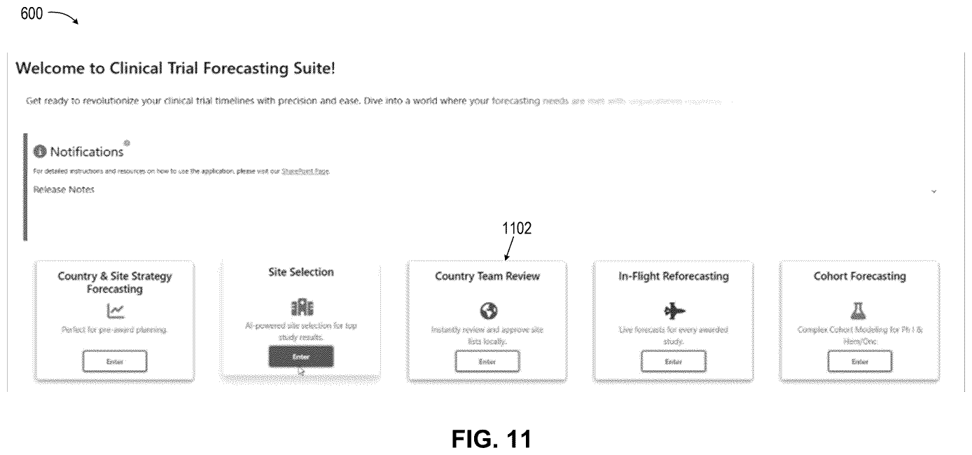

FIG. 10 illustrates a notification email 1000 generated by the site optimization module 305, according to some examples. In various implementations, the site optimization module 305 generates the notification email 1000 in response to one or more workflow events associated with the site-selection process, for example, after the selection of the save site list element 904. The notification email 1000 may be transmitted to relevant reviewers (for example, at respective user devices 112) and may present a study details field 1002 identifying key information about the study, as well as a hyperlink 1004 that directs the recipient to the country team review screen illustrated in FIG. 11. In various implementations, the site optimization module 305 populates the study details field 1002 according to the active site-list record stored within the forecasting system 110.

In some examples, reviewers may observe several types of notification emails throughout the workflow. A first type of notification may be transmitted when the strategist saves a site list as an active site list, thereby providing an initial alert that a new or updated set of sites has been created. A second type of notification may be transmitted when the strategist saves a site list with local team review workflow statuses applied to one or more sites, thereby informing regional or in-country reviewers that the list is available for examination. A third type of notification may be transmitted when a reviewer is assigned as country-level, local team, or designated reviewer for specific sites, and such an assignment notification may provide a direct-access link through the hyperlink 1004 to the country team review screen of FIG. 11. The content of each notification may instruct reviewers to observe particular identifiers associated with the study, such as a BC number (study identifier) referenced within the email text, for subsequent use within the relevant study-management system accessed by the reviewer.

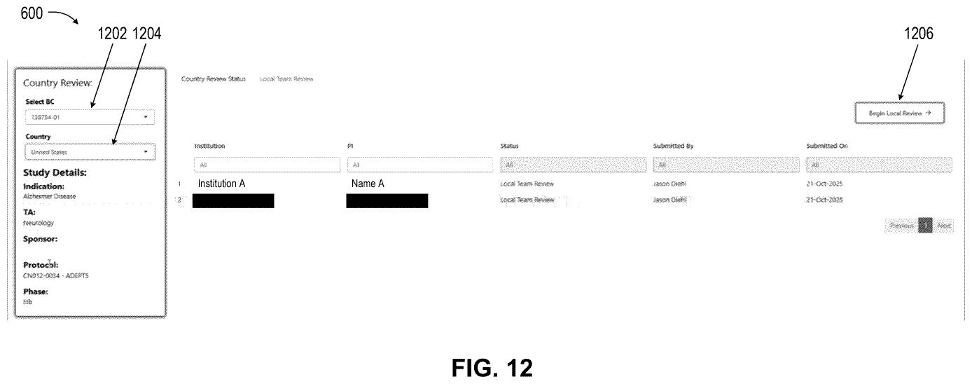

In various implementations, FIG. 11 illustrates a main screen of the clinical trial forecasting suite presented within the graphical user interface 600. This main screen may provide navigational access to multiple review and forecasting functions supported by the forecasting system 110. Among these functions, the graphical user interface may present a country team review element 1102 that allows a designated reviewer or strategist to initiate a geographic review workflow. The visualization module 310 may render this main screen in response to a navigation event generated from the notification email 1000 of FIG. 10 or in response to direct user interaction within the forecasting system 110. Selection of the country team review element 1102 may transition the user to the country team review screen depicted in FIG. 12.

In various implementations, FIG. 12 illustrates a country team review screen presented in response to selection of the country team review element 1102 on the main screen of FIG. 11. The country team review screen may allow a reviewer to specify a study identifier and country context for the local review phase of the workflow. The graphical user interface of FIG. 12 may present a select-identifier element 1202, which may appear as a drop-down menu, fillable field, or other selector through which the reviewer selects a reference identifier associated with the study. The graphical user interface may further present a country element 1204, which may appear as a drop-down menu or fillable field through which a reviewer specifies the country in which the local review is to be performed. The screen may also present a begin local review element 1206, which may appear as a selectable button or similar control used to initiate the reviewer-assignment process.

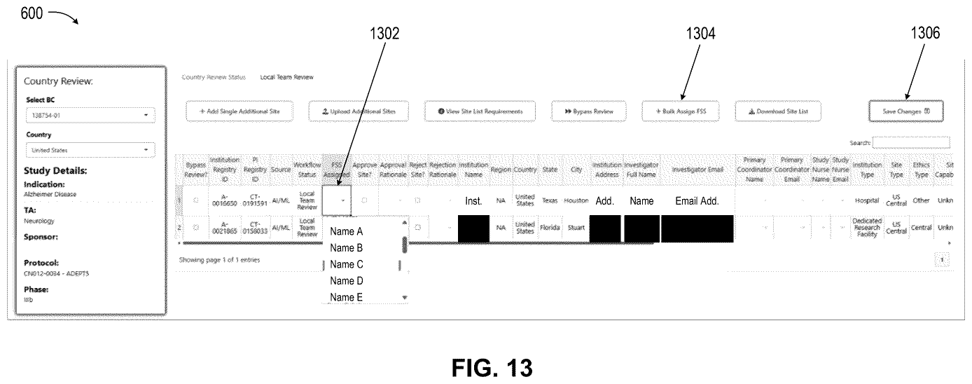

In some examples, once the reviewer selects values through the select-identifier element 1202 and the country element 1204, activation of the begin local review element 1206 may transition the user to the reviewer assignment screen illustrated in FIG. 13. At this stage, the forecasting system 110 may treat the selected identifier and country as context parameters for subsequent reviewer-assignment operations.

In various implementations, FIG. 13 illustrates a reviewer assignment screen rendered by the visualization module 310 in response to selection of the begin local review element 1206 of FIG. 12. This reviewer assignment screen may present an interface through which a strategist or designated reviewer assigns one or more review responsibilities for the sites associated with the study identifier and country selected on the preceding screen.

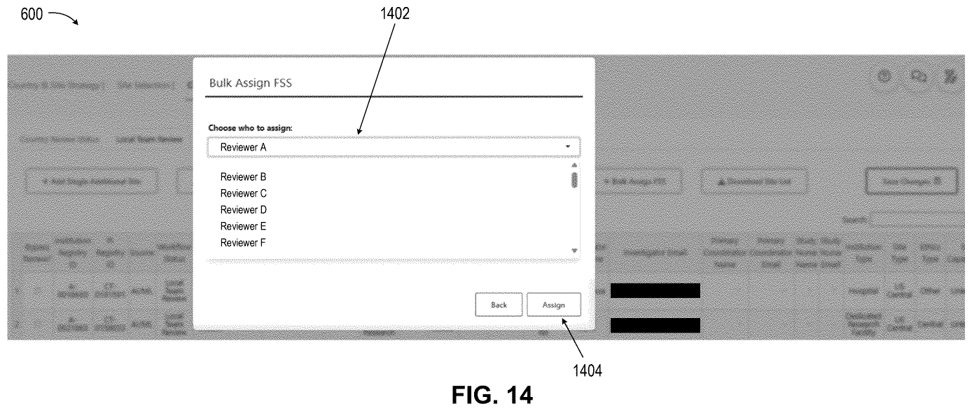

The graphical user interface 600 may display a reviewer assignment element 1302, which may appear as a drop-down menu or similar selector through which a reviewer is assigned to a particular site. In various implementations, the reviewer assignment element 1302 may present a list of eligible reviewers associated with the specified country or functional area. The reviewer assignment screen may further include a bulk assignment element 1304, which may appear as a selectable button or similar control allowing the user to initiate a bulk-assignment workflow for distributing multiple sites across multiple reviewers. Selection of the bulk assignment element 1304 may transition the graphical user interface 600 to the bulk assignment screen of FIG. 14.

In various implementations, FIG. 14 illustrates a bulk assignment screen generated in response to activation of the bulk assignment element 1304 on the reviewer assignment screen of FIG. 13. The bulk assignment screen may provide an interface through which a strategist selects one or more reviewers for distribution of site-review responsibilities. The graphical user interface 600 may display a drop-down element 1402 populated with a list of selectable reviewers eligible to perform the local review for the study. The strategist may select a single reviewer or multiple reviewers from the drop-down element 1402 to define the reviewer group for bulk distribution.

The bulk assignment screen may further include an assign element 1404, which may appear as a selectable button or similar control used to confirm the reviewer selections. In response to activation of the assign element 1404, the forecasting system 110 may apply a bulk-assignment procedure in which the site optimization module 305 allocates sites generally evenly across the reviewers selected through the drop-down element 1402. Upon completion of the assignment process, the graphical user interface 600 may return the user to the reviewer assignment screen of FIG. 13 for review and saving of the updated assignment configuration.

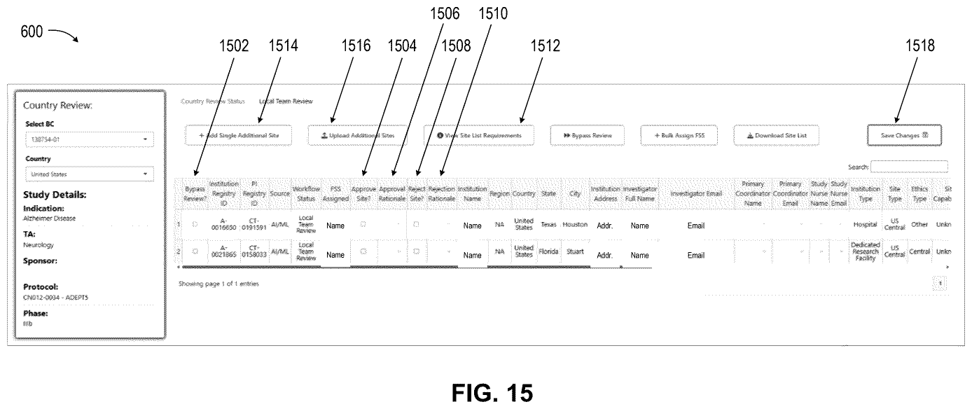

Upon completion of bulk-assignment operations, the graphical user interface may return the user to the reviewer assignment screen of FIG. 13. In some examples, activation of a save changes element 1306 on the reviewer assignment screen may commit the reviewer-assignment selections and transition the user to the workflow status screen illustrated in FIG. 15.

In various implementations, FIG. 15 illustrates a workflow status screen rendered by the visualization module 310 in response to saving the reviewer-assignment selections on the reviewer assignment screen of FIG. 13. The workflow status screen may allow a reviewer or strategist to evaluate each site included in the draft site list and provide a corresponding approval or rejection decision supported by one or more rationale inputs.

The workflow status screen may present a site table 1502 that displays multiple fields associated with each site under review. The site table 1502 may include, for example, a bypass-review column, one or more institutional registry-identifier fields, a source column indicating how the site was introduced into the list, a workflow-status column indicating whether a site is in draft or local-review status, a reviewer-assigned column, an approve-site column 1504 including selectable elements corresponding to individual sites, an approval-rationale column 1506 including fillable fields for entering one or more rationales supporting an approval decision, a reject-site column 1508 including selectable elements corresponding to individual sites, and a rejection-rationale column 1510 including fillable fields for entering one or more rationale values supporting a rejection decision. The site table 1502 may further display institutional and contact information including an institutional-name column, a region column, a country column, a state column, a city column, an institution-address column, an investigator full-name column, an investigator email column, a primary coordinator name column, a primary coordinator email column, a study nurse name column, a study nurse email column, an institution-type column, a site-type column, an ethics-type column, and a site-capabilities column.

In various implementations, the workflow-status column of the site table 1502 may indicate whether a site remains within a draft stage (during which the site list may still be under review by the strategist) or whether the site has transitioned to a local team review stage, in which the site is visible to the appropriate country-level reviewer. The reviewer may evaluate each row of the site table 1502 in view of the displayed institutional characteristics, investigator details, contact information, operational attributes, and study-specific metadata.

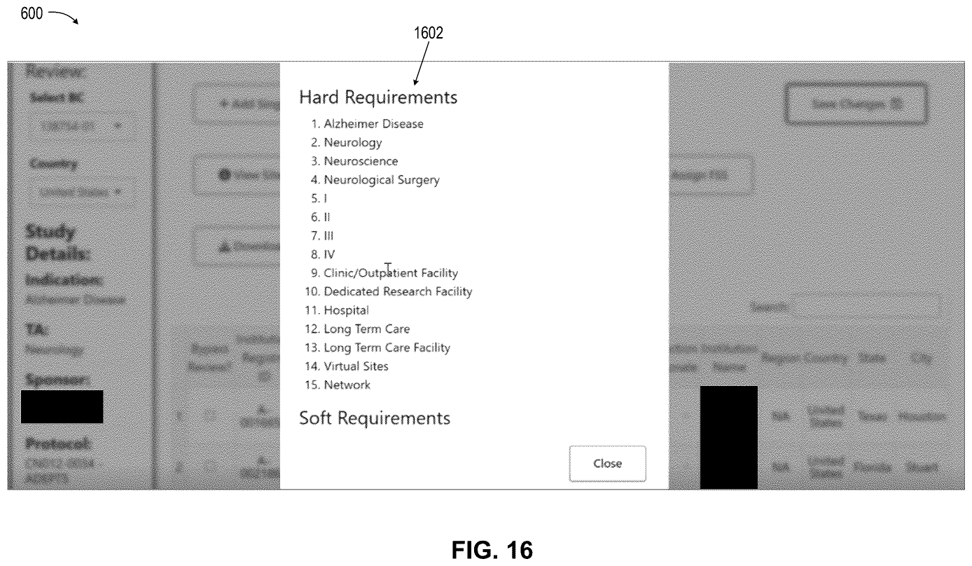

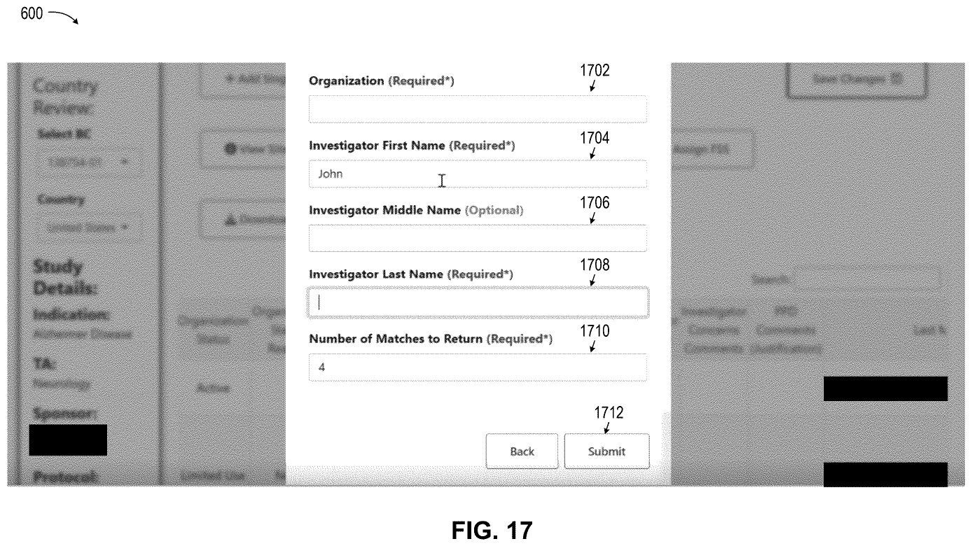

The workflow status screen may include a view site list requirements element 1512, which may appear as a selectable button or tab used to view criteria associated with the draft site list. Selection of the view site list requirements element 1512 may transition the graphical user interface 600 to the requirements screen illustrated in FIG. 16. In some examples, the workflow status screen may also include an add single additional site element 1514, which may allow the reviewer or strategist to introduce an additional site to the list. Activation of the add single additional site element 1514 may transition the graphical user interface 600 to the add-site screen of FIG. 17.

In response to reviewing the information presented in the site table 1502, a reviewer may select one or more rationale values in the approval-rationale column 1506 or the rejection-rationale column 1510 to justify the approval or rejection of each site. In some examples, rationale values may include attributes such as specialized investigator expertise, relevant indication experience, or access to an appropriate patient population. These rationale entries may be applied at an individual-site level or, in some implementations, may be applied across multiple sites as part of a bulk-selection workflow.

In various implementations, FIG. 16 illustrates a requirements screen generated in response to selection of the view site list requirements element 1512 on the workflow status screen of FIG. 15. The requirements screen may provide a structured summary of the hard requirements and soft requirements associated with the study for which the site list was constructed. The graphical user interface 600 may present a requirements list 1602 identifying parameters such as the indication, investigator specialty rules, study phase, institution-type selections, and other hard-criteria elements selected during the setup process illustrated in FIG. 6. In some examples, the requirements list 1602 may additionally include soft-criteria elements, such as applicable site capabilities, equipment considerations, or other contextual attributes derived from the soft-requirements workflow illustrated in FIG. 7.

The requirements screen may allow the reviewer to reference these criteria before returning to the workflow status screen of FIG. 15 to make approval or rejection determinations. In some examples, the forecasting system 110 utilizes the underlying criteria to provide visual cues or contextual guidance for the reviewer; however, the requirements list 1602 may serve primarily as an informational display to support consistent evaluation of sites during the local team review process.

In various implementations, FIG. 17 illustrates an add site screen generated in response to activation of the add single additional site element 1514 on the workflow status screen of FIG. 15. This add site screen may allow a reviewer or strategist to manually introduce a new site into the workflow by searching for investigator and institution records stored in an external registry system. The graphical user interface 600 may present one or more fillable fields through which the reviewer inputs identifying information for a known investigator-institution pair.

The add site screen may include a fillable organization field 1702 configured to receive an institution name or a wildcard-based search string. The add site screen may additionally provide a fillable investigator first-name field 1704, a fillable investigator middle-name field 1706, and a fillable investigator last-name field 1708, each of which may receive investigator-identifying information. A fillable number-of-matches-to-return field 1710 may permit the reviewer to specify how many potential matches the fuzzy-matching logic of the forecasting system 110 returns for evaluation. The add site screen may further include a submit element 1712, which may appear as a selectable button or similar control used to initiate a search request based on the fields populated by the reviewer.

In some examples, the forecasting system 110 may utilize fuzzy-matching logic to evaluate the text-based inputs received through the fields 1702-1710. This fuzzy-matching logic may return a ranked set of potential investigator-institution matches, such as the most relevant four to twenty matches identified from the underlying registry. The reviewer may then select the most accurate investigator and institution pair from the returned list before the forecasting system 110 incorporates the added site into the draft site list. The add site workflow of FIG. 17 may support search-based entry for investigators and institutions known to exist within the external registry.

To proceed with adding the site, the reviewer may input the investigator's first, middle, and last names, the institution name, and the ethics-type value selected from a drop-down menu associated with the add site workflow. The review may select the submit element 1712 to add the populated site.

In various implementations, the workflow status screen of FIG. 15 may further present an upload additional sites element 1516, which may appear as a selectable button or hyperlink used to initiate a batch-upload workflow for introducing additional investigator-institution records not available through the add site screen of FIG. 17. In some examples, activation of the upload additional sites element 1516 may provide access to a downloadable template, such as an Excel file, through which the reviewer or strategist enters investigator and institutional details for multiple new sites. Upon completion of the template, the reviewer may upload the populated file through the forecasting system 110 to introduce the additional sites into the draft site list.

In various implementations, the forecasting system 110 may utilize one or more error-checking procedures implemented by the site optimization module 305 to determine whether the uploaded investigator-institution combinations are already present within the draft site list. When a batch upload introduces a record with only partial overlap, such as an investigator already associated with a different institution within the list, the forecasting system 110 may create a new entry reflecting the investigator's association with the newly specified institution. When the batch upload includes a record that fully matches an existing entry, such as an investigator and institution pair already present in the draft list, the forecasting system 110 may refrain from generating a new row and instead associate an additional source designation with the existing record. In such examples, the workflow status screen may display one or more source identifiers indicating each internal or external contributor responsible for recommending the site.

The workflow status screen may additionally present a save changes element 1518, which may appear as a selectable button or similar control positioned near the bottom of the interface. In response to completing approval or rejection decisions across all assigned sites, the reviewer may activate the save changes element 1518 to submit all selections, rationale entries, and reviewer-assignment updates to the forecasting system 110. In various implementations, this action may update one or more workflow-status values associated with the affected sites and may prepare the site list for subsequent review stages or downstream forecasting operations.

In view thereof, it may be understood various embodiments of the trial planning system 10 disclosed herein may be configured to resolve one or more existing needs in the art. For instance, the modelling system 106 disclosed herein may enable accurate predictive trial output 228 for both the pre-award and post-award phases of a clinical trial, at least some of which, such as the post-award module, may be based on real-time data. Such a forecasting system 110, meanwhile, may enable a user to configure numerous parameters to adjust predictive trial output(s) 228 received from the modelling system 106, to both tailor and/or override such forecasts to the exact needs of a given trial. Such a forecasting system 110 may further utilize a visualization module 310 to overlay various predictive trial outputs 228 at varying levels of depth, thereby providing meaningful data to a user, and thus enabling a user to adjust a forecasted trial scenario at a granular level.

What is claimed is:

1. A system for clinical trial operational forecasting comprising:
a data warehouse communicatively configured in connection with at least one data source, a modelling system, and a forecasting system;
said data warehouse configured to receive a raw dataset from said at least one data source and generate at least one historical feature dataset from said raw dataset;
said modelling system configured to generate at least one predictive trial output according to said at least one historical feature dataset;
said forecasting system configured to receive said at least one predictive trial output and generate at least one visualization therefrom, said at least one visualization depicting a site activation curve and an enrollment curve.

2. The system of claim 1, wherein said at least one visualization comprises a study plot, a region plot, or a country plot.

3. The system of claim 1, wherein said modelling system comprises a pre-award module, said pre-award module configured to generate said predictive trial output related to a bid and planning phase of a clinical trial.

4. The system of claim 3, wherein said pre-award module comprises:
a qualification model configured to predict a qualification metric for at least one site;
an activation model configured to predict an activation metric for said at least one site;
an activated quantity model configured to predict an activated quantity metric for said at least one site;
a screening model configured to predict a screening metric for said at least one site;
a screen-fail model configured to predict a screen-fail metric for said at least one site; and
a dropout model configured to predict a dropout metric for said at least one site.

5. The system of claim 4, wherein said qualification model, said activation model, said activated quantity model, said screening model, said screen-fail model, and said dropout model each comprise a gradient boosted classification and regression tree model.

6. The system of claim 1, wherein said modelling system comprises a post-award module configured to generate said predictive trial output related to a post-award phase of a clinical trial.

7. The system of claim 6, wherein said post-award module comprises at least one model configured to predict a sequence of patient counts according to real-time data, wherein said at least one model comprises a sequence-based deep learning model.

8. The system of claim 1, wherein said forecasting system comprises a strategy forecasting module configured to receive, from a user, at least one consideration parameter and at least one milestone parameter.

9. The system of claim 8, wherein said at least one visualization comprises said at least one consideration parameter and said at least one milestone parameter.

10. A system for clinical trial operational forecasting comprising:
a data warehouse communicatively configured in connection with at least one data source, a modelling system, and a forecasting system;
said data warehouse configured to receive a raw dataset from said at least one data source;
said data warehouse comprising a historical feature module configured to generate an historical feature dataset from said raw dataset;
said modelling system comprising a pre-award module, said pre-award module comprising:
a qualification model configured to predict a qualification metric for at least one site;
an activation model configured to predict an activation metric for said at least one site;
an activated quantity model configured to predict an activated quantity metric for said at least one site;
a screening model configured to predict a screening metric for said at least one site;
a screen-fail model configured to predict a screen-fail metric for said at least one site;
a dropout model configured to predict a dropout metric for said at least one site;
said modelling system configured to generate at least one predictive trial output from said pre-award module;
said forecasting system configured to receive said at least one predictive trial output, said forecasting system comprising:
a strategy forecasting module configured receive at least one consideration parameter and at least one milestone parameter from at least one user device;
a reforecasting module configured to receive at least one patient diversity parameter from said at least one user device; and
a visualization module configured to generate at least one visualization according to said at least one consideration parameter, said at least one milestone parameter, said at least patient diversity parameter, and said at least one predictive trial output.

11. The system of claim 10, wherein said data warehouse further comprises a preprocessing module configured to generate at least one training dataset and at least one testing dataset from said historical feature dataset.

12. The system of claim 11, wherein said preprocessing module is further configured to generate at least one inference dataset according to at least one data distribution metric and at least one performance metric received by said data warehouse from said modelling system.

13. The system of claim 11, wherein said at least one training dataset and said at least one testing dataset are generated according to a train-test-split procedure.

14. The system of claim 11, wherein said modelling system further comprises a validation module, said validation module configured to train said qualification model, said activation model, said activated quantity model, said screening model, said screen-fail model, and said dropout model in parallel.

15. The system of claim 11, wherein said forecasting system further comprises an override module configured to receive at least one override parameter from said at least one user device, said at least one override parameter comprising study override data, start up override data, or enrollment override data.

16. The system of claim 11, wherein said forecasting system further comprises a site selection module, said site selection module comprising a catalog of a plurality of clinical trial sites.

17. The system of claim 16, wherein said site selection module further comprises at least one artificial intelligence algorithm configured to identify at least one site from said plurality of clinical trial sites according to said at least one consideration parameter, said at least one milestone parameter, and said at least one patient diversity parameter.

18. A system for clinical trial operational forecasting comprising:
a data warehouse communicatively configured in connection with at least one data source, a modelling system, and a forecasting system;
said data warehouse configured to receive a raw dataset from said at least one data source;
said data warehouse comprising a historical feature module configured to generate an historical feature dataset from said raw dataset;
said modelling system comprising a pre-award module and a post-award module;
said pre-award module comprising at least one pre-award model configured to generate at least one pre-award predictive trial output;
said post-award module comprising at least post-award model configured to generate at least one post-award predictive trial output, said at least one post-award predictive trial output comprising a sequence of patient counts for each month according to real-time data collected from said at least one data source;
said forecasting system configured to receive said at least one pre-award predictive trial output and said at least one post-award predictive trial output, said forecasting system comprising:
a visualization module configured to generate at least one visualization, said at least one visualization configured to overlay said at least one pre-award predictive trial output and said at least one post-award predictive trial output.

19. The system of claim 18, wherein said forecasting system comprises an override module configured to receive at least one override parameter from at least one user device, said at least one override parameter configured to alter said at least one pre-award predictive trial output.

20. The system of claim 18, wherein said at least one data source comprises a trial management system, an internal data source, and an external data source.
